(19) United States
(12) Patent Application Publication
(10) Pub. No.: US 2022/0251132 A1
(43) Pub. Date: Aug. 11, 2022

(54) LARGE SCALE PROCESS OF 3,3'-DIDEOXY-3,3'-BIS-[4-(3-FLUOROPHENYL)-1H-1,2,3-TRIAZOL-1-YL]-1,1'-SULFANEDIYL-DI-BETA-D-GALACTOPYRANOSIDE

(71) Applicant: GALECTO BIOTECH AB, COPENHAGEN (DK)

(72) Inventors: Fredrik ZETTERBERG, COPENHAGEN (DK); Andrew TYRELL, Reading (GB); Ulf NILSSON, COPENHAGEN (DK); Anders BERGH, Karlskoga (SE); Jonathan CUMMINS, COPENHAGEN (DK); Alexander WEYMOUTH-WILSON, Reading (GB); Robert CLARKSON, Reading (GB)

(73) Assignee: GALECTO BIOTECH AB, COPENHAGEN (DK)

(21) Appl. No.: 17/622,501

(22) PCT Filed: Jun. 24, 2020
(86) PCT No.: PCT/EP2020/067620

§ 371 Date: Dec. 23, 2021

(30) Foreign Application Priority Data
Jun. 24, 2019 (EP) 19181911.9

(51) Int. Cl. C07H 19/04; C07H 1/06

(52) U.S. Cl. CPC C07H 19/04; C07H 1/06

(57) ABSTRACT

The present invention relates to a process for preparing a compound of formula (I)

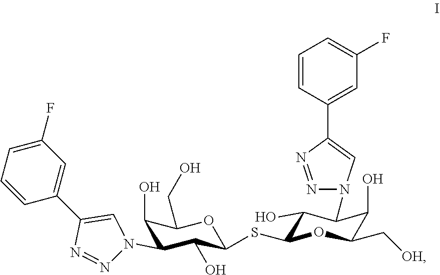

wherein said process is suitable for large scale synthesis.

TECHNICAL FIELD

The present invention relates to a process of preparing 3,3′-Dideoxy-3,3′-bis-[4-(3-fluorophenyl)-1H-1,2,3-triazol-1-yl]-1,1′-sulfanediyl-di-β-D- galactopyranoside which process can be scaled up. The process parameters are stable, and the process is suitable for GMP manufacture.

### BACKGROUND ART

Idiopathic pulmonary fibrosis (IPF) represents a massive worldwide health burden. It is a chronic condition of unknown etiology in which repeated acute lung injury causes progressive fibrosis resulting in destruction of lung architecture, deteriorating lung function with consequent respiratory failure and death. Although idiopathic pulmonary fibrosis (IPF) is the arche-type and most common cause of lung fibrosis, numerous respiratory diseases can progress to pulmonary fibrosis, and this usually signifies a worse prognosis. The median time to death from diagnosis is 2.5 years and the incidence and prevalence of IPF continues to rise. It remains one of the few respiratory conditions for which there are no effective therapies, and there are no reliable biomarkers to predict disease progression. The mechanisms resulting in pulmonary fibrosis are unclear but center around aberrant wound healing as a consequence of repetitive epithelia injury from an as yet unknown cause. IPF is characterized by fibroblastic foci containing fibroblasts/myofibroblasts which show increased activation response to fibro-genic cytokines such as transforming growth factor-β1 (TGF-β1). There is a big unmet need for drugs for treatment of Idiopathic pulmonary fibrosis. The compound 3,3′-Dideoxy-3,3′-bis-[4-(3-fluorophenyl)-1H-1,2,3-triazol-1-yl]-1,1′-sulfanediyl-di-β-D-galactopyranoside is currently in clinical phase II for treatment of IPF and a process of preparing the compound is described in WO2014/067986.

### SUMMARY OF THE INVENTION

The present invention relates to a new process for preparing 3,3′-Dideoxy-3,3′-bis-[4-(3-fluorophenyl)-1H-1,2,3-triazol-1-yl]-1,1′-sulfanediyl-di-β-D- galactopyranoside which can be upscaled to large scale and/or industrial scale such as 30 kg or higher, but also for smaller scale for instance from 2 kg to 30 kg, such as from 4 kg to 20 kg. The process can also be used for smaller scale such as from 200 g to 2 kg.

In a first aspect the present invention relates to a process, such as suitable for large scale synthesis, for preparing 3,3′-Dideoxy-3,3′-bis-[4-(3-fluorophenyl)-1H-1,2,3-triazol-1-yl]-1,1′-sulfanediyl-di-β-D-galactopyranoside having formula (I)

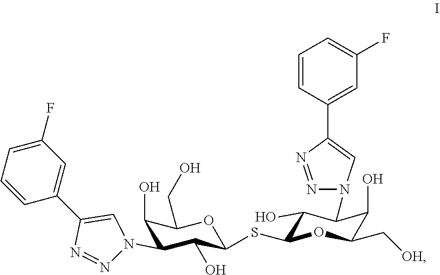

wherein the process comprises the consecutive steps of
a) reacting a compound of formula XVIII

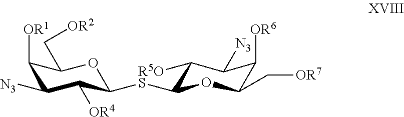

wherein R¹, R², R⁴, R⁵, R⁶ and R⁷ are independently selected from protecting groups or hydrogen, provided that at least one of R¹, R², R⁴, R⁵, R⁶ and R⁷ is a protecting group, with 3-fluoro-phenyl acetylene or a 3-fluorophenyl acetylene protected at the terminal position of the acetylene group, under suitable conditions to obtain a compound of formula XIX

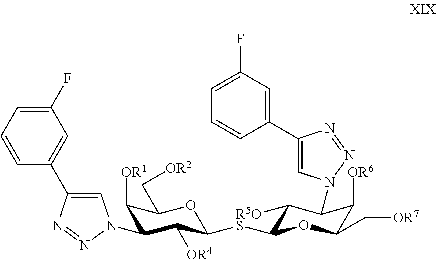

wherein R¹, R², R⁴, R⁵, R⁶ and R⁷ are as defined above, and b) removing the protecting groups of the compound of formula XIX to obtain the compound of formula I.

In one embodiment, the compound of formula I is purified and isolated, such as isolated as a solid product, such as a crystalline or amorphous product.

In a further embodiment the suitable conditions in step a) are reacting a compound of formula XVIII wherein R1, R2, R4, R5, R6 and R7 are independently selected from protecting groups or hydrogen, provided that at least one of R1, R2, R4, R5, R6 and R7 is a protecting group, and a catalyst in an organic solvent optionally under inert atmosphere and at a suitable temperature with 3-fluorophenyl acetylene and optionally a base in the organic solvent to create a reaction mixture and optionally heating the reaction mixture to raise the temperature at least 15° C. above the suitable temperature and continue the reaction for at least 1 hour to obtain a compound of formula XIX wherein R1, R2, R4, R5, R6 and R7 are as defined above.

In another embodiment the suitable conditions in step a) are reacting a compound of formula XVIII wherein R1, R2, R4, R5, R6 and R7 are independently selected from protecting groups or hydrogen, provided that at least one of R1, R2, R4, R5, R6 and R7 is a protecting group, and a catalyst in an organic solvent optionally under inert atmosphere and at a suitable temperature with a 3-fluorophenyl acetylene protected at the terminal position of the acetylene group and optionally a base in the organic solvent to create a reaction mixture and optionally heating the reaction mixture to raise the temperature at least 15° C. above the suitable temperature and continue the reaction for at least 1 hour to obtain a compound of formula XIX wherein R1, R2, R4, R5, R6 and R7 are as defined above. Typically, the terminal position (C-H) of the acetylene group is protected with a protecting group such as a carboxyl group, a Cu, a Ag, a silyl protecting group, for instance trimethyl silyl, tert-butyldimethyl silyl, triethyl silyl, triphenyl silyl, tert-butyldiphenyl silyl.

Throughout this text 3-fluorophenyl acetylene and a 3-fluorophenyl acetylene protected at the terminal position of the acetylene group are considered to be independent embodiments.

In a still further embodiment R1, R2, R4, R5, R6 and R7 are independently selected from ester protecting groups such as acetyl, benzoyl and pivaloyl, typically all R1, R2, R4, R5, R6 and R7 are identical, such as acetyl.

A further embodiment further comprises purifying and isolating the compound of formula XIX as a solid.

In a further embodiment the reaction takes place under inert atmosphere, such as an argon or nitrogen atmosphere.

In a still further embodiment the organic solvent is selected from toluene or a polar aprotic solvent, such as ethyl acetate, acetonitrile or DMF, and mixtures thereof.

In a further embodiment the suitable temperature is between 15 and 25° C., such as about room temperature.

In a still further embodiment the temperature is raised in the reaction mixture heating the mixture to 45° C. to 60° C., such as about 50° C.

In a further embodiment the reaction is continued for at least 2 hours, such as 3 hours, e.g. from 2.5 to 4 hours.

In a still further embodiment the catalyst is a metal catalyst, such as a metal halide, e.g. Cu(I) or Cu(II), in particular Cu halide, such as Cu iodide.

In a further embodiment the base is an organic base, such as an aliphatic amine base, e.g. triethylamine or N,N-diisopropylethylamine (DIPEA), typically triethylamine.

In a still further embodiment the protecting groups are removed in step b) by mixing the compound of formula XIX with a base and reacting for at least 15 minutes at a suitable temperature, followed by neutralizing with a suitable acid in solution to obtain the compound of formula I. Typically, the heating of the suspension is to at least 60° C., such as 60-90° C., e.g. about 75° C. Preferably, the base is in an organic solvent, such as a C₁₋₆ alcohol, typically methanol as well as mixtures thereof. Preferably, the base is selected from a base, such as an organic base, in a concentration sufficient to remove the protecting groups, such as a base at a pH of 12 or higher. Typically, the base is sodium methoxide in methanol, such as 25 wt % sodium methoxide solution in methanol. Preferably, the reaction with a base is for 20-60 minutes, such as about 30 minutes. Preferably, the suitable temperature is 15-25° C., such as about room temperature. Typically, the neutralizing acid in solution is aqueous HCl.

In a further embodiment the molar ratio between the compound of formula XVIII and 3-fluorophenyl acetylene is 1:5 to 1:1, such as 1:4 to 1:2, e.g. 1:3 and the organic solvent is in surplus. In a further embodiment the molar ratio between the compound of formula XVIII and the catalyst is 3:1 to 7:1, such as 4:1 to 6:1, e.g. 5:1 and the organic solvent is in surplus. Further embodiments cover the molar ratio between the compound of formula XVIII and the base is 1:2 to 4:1, such as 1:1 to 3:1, e.g. 2:1 and the organic solvent is in surplus.

In a further embodiment the molar ratio between the compound of formula XVIII and a 3-fluorophenyl acetylene protected at the terminal position of the acetylene group, is 1:5 to 1:1, such as 1:4 to 1:2, e.g. 1:3 and the organic solvent is in surplus. In a further embodiment the molar ratio between the compound of formula XVIII and the catalyst is 3:1 to 7:1, such as 4:1 to 6:1, e.g. 5:1 and the organic solvent is in surplus. Further embodiments cover the molar ratio between the compound of formula XVIII and the base is 1:2 to 4:1, such as 1:1 to 3:1, e.g. 2:1 and the organic solvent is in surplus.

In a still further embodiment the process comprises a step directly preceding step a)
(i) reacting a compound of formula XVII

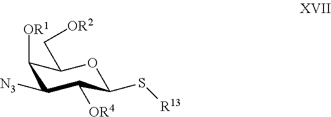

wherein R1, R2, and R4 are independently selected from protecting groups or hydrogen, provided that at least one of R1, R2, and R4 is a protecting group, and R13 is H or a protecting group, such as a silyl group, e.g. triisopropylsilyl, a triflate group, acetyl, or C(═NH)—NH₂, with a compound of formula XX

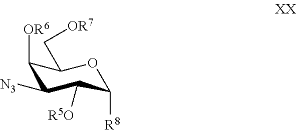

wherein R5, R6 and R7 are independently selected from protecting groups or hydrogen, provided that at least one of R5, R6 and R7 is a protecting group, and R8 is a halogen, SR10 or OR10 wherein R10 is selected from H, Z-C₁₋₆ alkyl, Z-C₂₋₆alkenyl, Z-C₃₋₆branched alkyl, Z-C₃₋₆ cyclo alkyl, Z-heteroaryl, and Z-aryl wherein Z is SO, SO₂, C═O or C═S, under suitable conditions to obtain the compound of formula XVIII

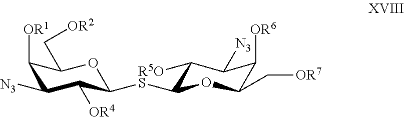

wherein R1, R2, R4, R5, R6 and R7 are independently selected from protecting groups or hydrogen, provided that at least one of R1, R2, R4, R5, R6 and R7 is a protecting group. In further embodiments the compound of formula XVIII is obtained as a solid product, such as a crystalline or amorphous product.

In a further embodiment the suitable conditions in step (i) are reacting a compound of formula XVII wherein R1, R2, and R4, are as defined above, in a first organic solvent optionally under inert atmosphere and at a suitable temperature with a solution of the compound of formula XX wherein R5, R6 R7, R8 and R10 are as defined above in a second organic solvent and adding a base optionally together with a third organic solvent to create a reaction mixture which is kept at a suitable temperature for a time sufficient to complete reaction and hereafter the mixture is concentrated, such as in vacuo at 40° C., to create a residue which is suspended in a fourth organic solvent at a second suitable temperature together with an aqueous acid solution and stirred to obtain a compound of formula XVIII, and optionally isolating and purifying to obtain a solid compound of formula XVIII. Typically, R1, R2, R4, R5, R6 and R7 are all identical groups, such as acetyl groups and R8 is as defined above. Preferably, R8 is a halogen, such as bromine.

In a still further embodiment the reaction takes place under inert atmosphere, such as an argon or nitrogen atmosphere.

In a further embodiment the first organic solvent is a polar aprotic solvent, such as acetonitrile, ethyl acetate, toluene, or DMF, especially acetonitrile.

In a still further embodiment the suitable temperature is between 15 and 25° C., such as about room temperature.

In a further embodiment the second organic solvent is a polar aprotic solvent such as toluene, ethyl acetate, acetonitrile or DMF, especially acetonitrile.

In a still further embodiment the base is an organic base selected from an aliphatic amine base, such as triethylamine and N,N-Diisopropylethylamine (DIPEA), typically triethylamine, and the optional third organic solvent is selected from the group consisting of ethyl acetate, toluene, DMF and acetonitrile, typically acetonitrile.

In a further embodiment the reaction is continued for at least 2 hours, such as at least 12 hours, e.g. at least 16 hours, such as 18 hours wherein the suitable temperature is 15-25° C. Preferably, the reaction mixture is concentrated in vacuo at 40° C. to create a residue, wherein the residue is suspended in ethyl acetate and the second suitable temperature is 15-25° C. together with an aqueous HCl solution, such as 2M HCl solution, and stirred to obtain a compound of formula XVIII.

In a still further embodiment the compound of formula XVIII is isolated and purified to obtain the solid compound of formula XVIII.

In a further embodiment the molar ratio between the compound of formula XVII and XX is 1:2 to 2:1, such as 2:3 to 3:2, e.g. 1:1, and the organic solvent is in surplus.

In a still further embodiment the process comprises a step directly preceding step i)
(ia) reacting a compound of formula XVI

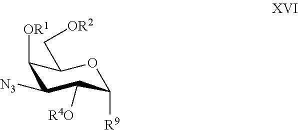

wherein R1, R2, and R4 are independently selected from protecting groups or hydrogen, provided that at least one of R1, R2, and R4 is a protecting group, and R9 is a halogen, SR11 or OR11 wherein R11 is selected from H, Z′-C₁₋₆ alkyl, Z′-C₂₋₆ alkenyl, Z′-C₃₋₆ branched alkyl, Z′-C₃₋₆ cyclo alkyl, Z′-heteroaryl and Z′-aryl wherein Z′ is SO, SO₂, C═O or C═S, with a reagent introducing a thiol or thiol linked to a protective group, such as, but not limited to, TIPS-SH, Na₂S, or thiourea, under suitable conditions to obtain the compound of formula XVII wherein R1, R2, and R4 are independently selected from protecting groups or hydrogen, provided that at least one of R1, R2, and R4 is a protecting group.

In an embodiment the compound of formula XVII is obtained as a solid product, such as a crystalline or amorphous product.

In a further embodiment the suitable conditions in step (ia) are reacting a compound of formula XVI wherein R1, R2, R4 and R9 are as defined above, in a first organic solvent optionally under inert atmosphere and at a suitable temperature with a solution of thiourea in a second organic solvent, then heating the reaction mixture to a temperature being at least 30° C. higher than the suitable temperature and continue reaction for at least 3 hours followed by cooling to a temperature at least 10° C. lower than the suitable temperature, and continue reaction for at least 1 hour, such as at least 3 hours, and optionally isolating and purifying to obtain a solid compound of formula XVII.

In a still further embodiment R1, R2, R4 are all acetyl groups and R9 is as defined above. Preferably, R9 is a halogen, such as bromine.

In a further embodiment the reaction takes place under inert atmosphere, such as an argon or nitrogen atmosphere.

In a still further embodiment the first organic solvent is selected from the group consisting of ethyl acetate, toluene, DMF and acetonitrile, typically acetonitrile.

In a further embodiment the suitable temperature is between 15 and 25° C., such as about room temperature.

In a still further embodiment the second organic solvent is selected from the group consisting of ethyl acetate, toluene, DMF and acetonitrile, typically acetonitrile.

In a further embodiment the reaction mixture is heated to a temperature being at least 60° C., such as 80° C.

In a still further embodiment the reaction is continued for at least 4 hours, such as 5 hours, following heating the reaction mixture.

In a further embodiment the reaction mixture is cooled to a temperature between 0° C. and 15° C., such as about 5° C.

In a still further embodiment the reaction is continued for at least 4 hours, such as 5 hours, following cooling of the reaction mixture.

In a further embodiment the compound of formula XVII is isolated and purified to obtain the solid compound of formula XVII.

In a still further embodiment the molar ratio between the compound of formula XVI and thiourea is 1:2 to 2:1, such as 2:3 to 3:2, e.g. 1:1, and the organic solvent is in surplus.

In a further embodiment the process comprises a step directly preceding step ia) (ib) reacting a compound of formula XV

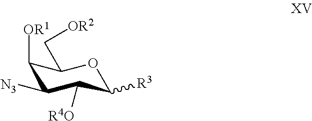

wherein the wavy line indicates that R3 is anomeric alpha, beta or a mixture of alpha and beta, wherein R1, R2, and R4 are independently selected from protecting groups or hydrogen, provided that at least one of R1, R2 and R4 is a protecting group, and R3 is a halogen, SR12 or OR12 wherein R12 is selected from H, Z″-C₁₋₆ alkyl, Z″-C₂₋₆ alkenyl, Z″-C₃₋₆branched alkyl, Z″-C₃₋₆ cyclo alkyl Z″-heteroaryl and Z″-aryl wherein Z″ is SO, SO₂, C═O or C═S, in a first suitable organic solvent with a reagent for activating the anomeric position for nucleophilic substitution, such as a halogenating agent or triflate, in a second suitable organic solvent under suitable conditions to obtain the compound of formula XVI wherein R1, R2, and R4 are independently selected from protecting groups or hydrogen, provided that at least one of R1, R2, and R4 is a protecting group and R9 is a halogen, SR11 or OR11 wherein R11 is selected from H, Z′-C₁₋₆ alkyl, Z′-C₂₋₆ alkenyl, Z′-C₃₋₆ branched alkyl, Z′-C₃₋₆ cyclo alkyl, Z′-heteroaryl and Z′-aryl wherein Z′ is SO, SO₂, C═O or C═S. Typically, the compound of formula XVI is obtained as a liquid product, such as an oil.

In a further embodiment the suitable conditions in step (ib) are reacting a compound of formula XV wherein R1, R2, R3 and R4, are as defined above, in a first organic solvent optionally under inert atmosphere and at a suitable temperature with the halogenating agent in a sec- and organic solvent, and maintaining the reaction mixture at the suitable temperature, then continue the reaction for at least 1 hour, such as 14 hours, followed by cooling to a temperature at least 10° C. lower than the suitable temperature, and optionally isolating and purifying to obtain the compound of formula XVII as an oil.

In a still further embodiment R1, R2, R4 are all acetyl groups and R3 is as defined above. Preferably, R3 is OR12 wherein R12 is selected from Z″-C₁₋₆ alkyl, Z″-C₂₋₆ alkenyl, Z″-C₃₋₆ branched alkyl, Z″-C₃₋₆ cyclo alkyl Z″-heteroaryl and Z″-aryl wherein Z″ is SO, SO₂, C═O or C═S, such as acetyl.

In a further embodiment the reaction takes place under inert atmosphere, such as an argon atmosphere. In a further embodiment the first organic solvent is selected from the group consisting of ethyl acetate, toluene, DMF and acetonitrile, such as ethyl acetate.

In a still further embodiment the suitable temperature is between 15° C. and 70° C., such as from 20° C. to 65° C. In a further embodiment the suitable temperature is between 25 ° C. and 60° C., such as between 30° C. and 50° C.

In a further embodiment the second organic solvent is selected from the group consisting of ethyl acetate, toluene, DMF and acetonitrile, such as ethyl acetate.

In a still further embodiment the halogenating agent is selected from the group consisting of BiBr₃, TMSBr, HBr and TiBr₄.

In a further embodiment the suitable temperature is maintained under 60° C.

In a still further embodiment the reaction is continued for at least 2 hours, such as from 3 to 20 hours, such as from 4 to 10 hours, at the suitable temperature. In another embodiment the reaction is continued for at least 3 hours, such as from 3 to 6 hours, such as from 3 to 4 hours, at the suitable temperature, such as between 30° C. and 50° C., e.g. between 40° C. and 45° C.

In a further embodiment the reaction mixture is cooled to a temperature between 0° C. and 15° C., such as about 5° C. In another embodiment the reaction mixture is cooled to a temperature between 5° C. and 25° C., such as about 20° C.

In a still further embodiment the compound of formula XVI is isolated and purified to obtain the oil compound of formula XVI.

In a further embodiment the molar ratio between the compound of formula XV and the halogenating agent is 1:3 to 3:1, such as 1:2 to 1:1, e.g. 3:4 and the organic solvent is in surplus.

In a second aspect the present invention relates to a compound of formula XVIII

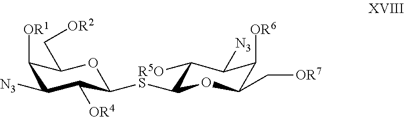

wherein R1, R2, R4, R5, R6 and R7 are independently selected from protecting groups or hydrogen, provided that at least one of R1, R2, R4, R5, R6 and R7 is a protecting group.

In a third aspect the present invention relates to a compound of formula XIX

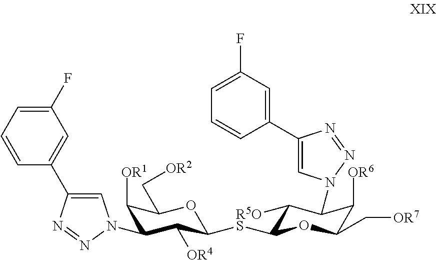

wherein R1, R2, R4, R5, R6 and R7 are independently selected from protecting groups or hydrogen, provided that at least one of R1, R2, R4, R5, R6 and R7 is a protecting group.

In a fourth aspect the present invention relates to a compound of formula XVII

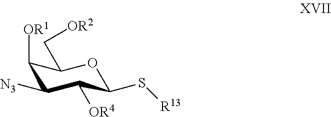

wherein R1, R2, and R4 are independently selected from protecting groups or hydrogen, provided that at least one of R1, R2, and R4 is a protecting group, and R13 is H or a protecting group, such as a silyl group, e.g. triisopropylsilyl, a triflate group, acetyl, or C(═NH)—NH₂.

In a fifth aspect the present invention relates to a compound of formula XX

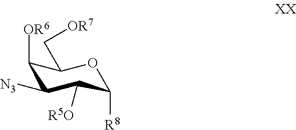

wherein R5, R6 and R7 are independently selected from protecting groups or hydrogen, provided that at least one of R5, R6 and R7 is a protecting group, and R8 is a halogen, SR10 or OR10 wherein R10 is selected from H, Z-C₁₋₆ alkyl, Z-C₂₋₆alkenyl, Z-C₃₋₆branched alkyl, Z-C₃₋₆ cyclo alkyl, Z-heteroaryl, and Z-aryl wherein Z is SO, SO₂, C═O or C═S.

In a sixth aspect the present invention relates to a compound of formula XVI

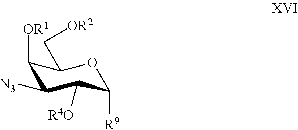

wherein R1, R2, and R4 are independently selected from protecting groups or hydrogen, provided that at least one of R1, R2, and R4 is a protecting group, and R9 is a halogen, SR11 or OR11 wherein R11 is selected from H, Z′-C₁₋₆ alkyl, Z′-C₂₋₆ alkenyl, Z′-C₃₋₆ branched alkyl, Z′-C₃₋₆ cyclo alkyl, Z′-heteroaryl and Z′-aryl wherein Z′ is SO, SO₂, C═O or C═S.

In a seventh aspect the present invention relates to a compound of formula XV

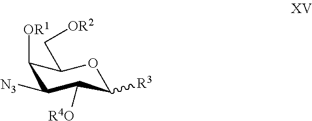

wherein the wavy line indicates that R3 is anomeric alpha, beta or a mixture of alpha and beta, wherein R1, R2, and R4 are independently selected from protecting groups or hydrogen, provided that at least one of R1, R2 and R4 is a protecting group, and R3 is a halogen, SR12 or OR12 wherein R12 is selected from H, Z″-C₁₋₆ alkyl, Z″-C₂₋₆ alkenyl, Z″-C₃₋₆branched alkyl, Z″-C₃₋₆ cyclo alkyl Z″-heteroaryl and Z″-aryl wherein Z″ is SO, SO₂, C═O or C═S.

### DETAILED DESCRIPTION

The compound of formula (I) has the chemical name (IUPAC) 3,3′-Dideoxy-3,3′-bis-[4-(3-fluorophenyl)-1H-1,2,3 -triazol-1-yl]-1,1′-sulfanediyl-di-β- D-galactopyranoside.

The compound of formula (I) may be prepared as described in US2014/0121179 or WO2014/067986, wherein an amorphous solid is produced.

The process of the present invention is illustrated in the scheme below starting from compound of formula XV and going through steps b9 to b13 to make the compound of formula I, and the process is up scalable to large scale and industrial scale.

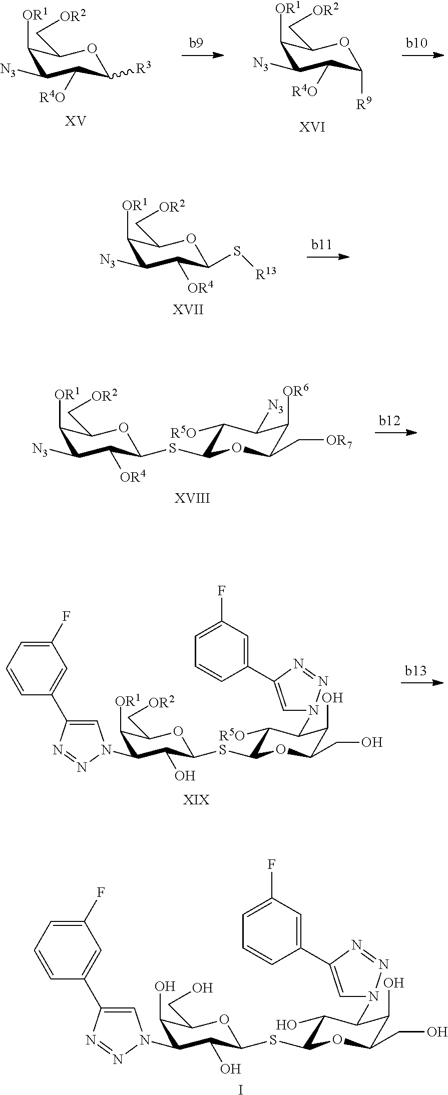

The above intermediates XV, XVI, XVII, XVIII and XIX may be formed in situ or may be isolated during the reaction process and if suitable salts thereof may be used during the present process, and such salts are covered by the above structures XV, XVI, XVII, XVIII and XIX. In further embodiments each of the above structures XV, XVI, XVII, XVIII and XIX are isolated as a salt thereof, such as a HCl or HBr salt. The step b13 is a deprotecting step to prepare the compound of formula I. The compound of formula XIX

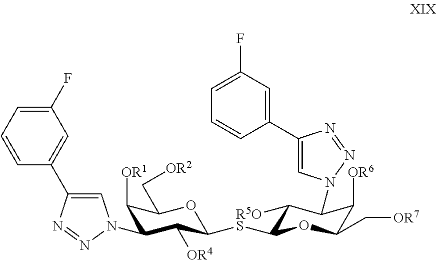

Which has several protecting groups R1, R2, R4, R5, R6 and R7 which may be removed by processes known to the person skilled in the art to obtain the compound of formula I. Not all groups R1-R7 needs to be protecting groups but at least one of R1-R7 is a protecting group and the rest is either hydrogen or a protecting group. The protecting groups are independently selected from ester protecting groups such as acetyl, benzoyl and pivaloyl, typically all R1, R2, R4, R5, R6 and R7 are identical. In a typical embodiment all R1-R7 are acetyl. The removing of protecting groups in step b13 is preferably done by mixing the compound of formula XIX with a base in an organic solvent and reacting for at least 15 minutes at a suitable temperature, followed by neutralizing with a suitable acid in solution to obtain the compound of formula I. A typical reaction is mixing the compound of formula XIX with base in a concentration sufficient to provide a pH of 12 or higher in an organic solvent selected from toluene or a polar aprotic solvent and reacting for 20-60 minutes, at a temperature from 60-90° C. followed by neutralizing with a suitable acid, such as aqueous hydrochloric acid, to obtain the compound of formula I.

The step b12 is a step for introducing the triazols and fluoro (F) substituted phenyl by chemically modifying the azide groups in the compound of formula XVIII

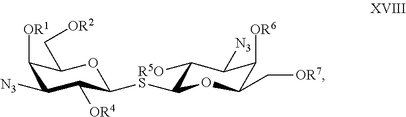

wherein R1-R7 are independently selected from protecting groups or hydrogen, provided that at least one of R1, R2, R4, R5, R6 and R7 is a protecting group. The compound of formula XVIII is reacted with 3-fluorophenyl acetylene or a 3-fluorophenyl acetylene protected at the terminal position of the acetylene group, under suitable conditions to obtain the compound of formula XIX. Preferably, the suitable condition is the use of a catalyst in an organic solvent. If the reaction is run without a catalyst then heating of the mixture is necessary. The catalyst is preferably a metal catalyst. Individual embodiments are selected from one or more of a metal halide, e.g. Cu(I) or Cu(II), in particular Cu halide, such as Cu iodide. The organic solvent can be any solvent such as toluene or a polar aprotic solvent. In an embodiment the solvent is selected from toluene, acetonitrile and DMF, and mixtures thereof. The reaction may be carried out in atmospheric air; however, this is not preferable due to possible poisoning of the catalyst. Typically, the reaction is carried out under inert atmosphere such as nitrogen or argon, preferably nitrogen. The suitable temperature preferably is between 15 and 25° C. Typically, about room temperature, such as between 18 and 22° C. In an embodiment no base is present, however, this is not preferable due to the reaction running slower without the base. Typically, a base is present in the organic solvent during the reaction, such as an organic base. Preferably the organic base is selected from an aliphatic amine base, such as triethylamine and N,N-Diisopropylethylamine (DIPEA), typically triethylamine. Typically, the reaction mixture containing the compound of formula XVIII, 3-fluorophenyl acetylene or a 3-fluorophenyl acetylene protected at the terminal position of the acetylene group, organic solvent, a catalyst and under inert atmosphere is heated to raise the temperature at least 15° C. above the suitable temperature and continue the reaction for at least 1 hour to obtain a compound of formula XIX. Preferably the reaction mixture is heated from a temperature between 15 and 25° C. to a temperature of at least 30° C., such as at least 40° C. In further embodiments the temperature is heated to a temperature between 45° C. to 60° C., such as about 50° C. The reaction is continued to at this higher temperature for a sufficient time to prepare the compound of formula XIX in a suitable yield, such as at least 1 hour, such as at least 2 hours, such as at least 3 hours, e.g. from 1 to 4 hours. In the reaction mixture the molar ratio between the compound of formula XVIII and 3-fluorophenyl acetylene or a 3-fluorophenyl acetylene protected at the terminal position of the acetylene group, is typically from 1:5 to 1:2, such as 1:4 to 1:2, e.g. 1:3 and the organic solvent is in surplus. In a further embodiment the molar ratio between the compound of formula XVIII and the catalyst is 3:1 to 10:1, such as 4:1 to 6:1, e.g. 5:1 and the organic solvent is in surplus. In a still further embodiment the molar ratio between the compound of formula XVIII and the base is 1:2 to 4:1, such as 1:1 to 3:1, e.g. 2:1 and the organic solvent is in surplus.

The step b11 is a step for preparing the compound of formula XVIII

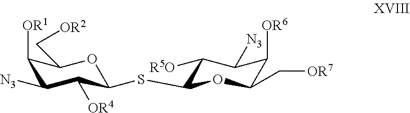

Wherein R1-R7 are as defined above, including the various embodiments. The compound of formula XVII

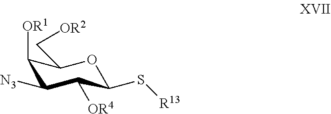

wherein R1, R2, and R4 are independently selected from protecting groups or hydrogen, provided that at least one of R1, R2, and R4 is a protecting group, and R13 is H or a protecting group, such as a silyl group, e.g. triisopropylsilyl, a triflate group, acetyl, or C(═NH)—NH₂, is reacted with a compound of formula XX

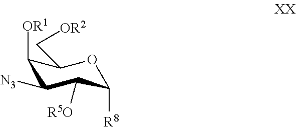

wherein R5, R6 and R7 are independently selected from protecting groups or hydrogen, provided that at least one of R5, R6 and R7 is a protecting group, and R8 is a halogen, SR10 or OR10 wherein R10 is selected from H, Z-C₁₋₆ alkyl, Z-C₂₋₆alkenyl, Z-C₃₋₆branched alkyl, Z-C₃₋₆ cyclo alkyl, Z-heteroaryl, and Z-aryl wherein Z is SO, SO₂, C═O or C═S, in a first organic solvent optionally under inert atmosphere and at a suitable temperature with a solution of the compound of formula XX wherein R5, R6 R7, R8 and R10 are as defined above in a second organic solvent and adding a base optionally together with a third organic solvent to create a reaction mixture which is kept at a suitable temperature for a time sufficient to complete reaction and hereafter the mixture is concentrated to create a residue which is suspended in a fourth organic solvent at a second suitable temperature together with an aqueous acid solution and stirred to obtain a compound of formula XVIII, and optionally isolating and purifying to obtain a solid compound of formula XVIII. Preferably, in the compound of formula XX R5, R6 and R7 are all identical groups, such as acetyl groups and R8 is a halogen, such as bromine. Preferably, in the compound of formula XVII R1, R2, R4 are all identical groups, such as acetyl groups and R13 is C(═NH)—NH₂. Preferably, the reaction takes place under inert atmosphere, such as a nitrogen atmosphere. The first, second, third and fourth organic solvent may be the same or different and are preferably selected from polar aprotic solvents. A preferred first, second, third and fourth organic solvent are independently selected from acetone, DMSO, THF, toluene, ethyl acetate, acetonitrile or DMF. Preferably, the suitable temperature is between 15 and 25° C., such as between 18 and 22° C. Typically, the base is an organic base, such as triethylamine, DIPEA, Lithium disopropylamide (LDA), sodium hexamethyldisilazane (NaHMDS), pyridine, DMAP, typically triethylamine, and the third organic solvent is acetonitrile. Preferably, the reaction is continued for at least 2 hours, such as at least 12 hours, e.g. at least 16 hours, such as 18 hours, for instance between 2 and 18 hours. Preferably, the reaction mixture is concentrated in vacuo from 30-50° C., such as about 40° C. to create a residue. The residue is suspended in the fourth organic solvent, such as ethyl acetate, toluene, DMF or acetonitrile, typically ethyl acetate, and the second suitable temperature is 15-25° C. such as between 18 and 22° C. Preferably, the aqueous acid solution is sulphuric acid or hydrochloric acid in water, such as 2M HCl solution. Typically, in the reaction mixture the molar ratio between the compound of formula XVII and XX is 1:2 to 2:1, in a further embodiment 2:3 to 3:2, such as about. 1:1, and the organic solvent is in surplus.

The step b10 is a step for introducing the sulphur (thio) in the anomeric beta position from the compound of formula XVI

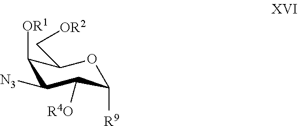

wherein R1, R2, R4 are as defined above, including the various embodiments, and R9 is as defined above to obtain the compound of formula XVII. The compound of formula XVI is reacted with a reagent introducing a thiol or thiol linked to a protective group, such as, but not limited to, TIPS-SH, Na₂S, or thiourea, under suitable conditions to obtain the compound of formula XVII. Preferably the thio introducing agent is S=R13, such as thiourea. Typically, when thiourea is used R9 is a halogen, such as bromine. Preferably, R1, R2, R4 are all protecting groups, in particular acetyl. Preferably the suitable conditions are reacting a compound of formula XVI wherein R1, R2, R4 and R9 are as defined above, in a first organic solvent optionally under inert atmosphere and at a suitable temperature with a solution of thiourea in a second organic solvent, then heating the reaction mixture to a temperature being at least 30° C. higher than the suitable temperature and continue reaction for at least 3 hours followed by cooling to a temperature at least 10° C. lower than the suitable temperature, and continue reaction for at least 1 hour. Preferably, the reaction takes place under inert atmosphere, such as an argon or nitrogen atmosphere, preferably nitrogen. The first and second organic solvent may be the same or different and are typically selected from polar aprotic solvents. A preferred first and second organic solvent is toluene, ethyl acetate, acetonitrile or DMF. Preferably the first and second solvent is acetonitrile. Preferably, the suitable temperature is between 15 and 25° C., such as between 18 and 22° C. When the suitable temperature is between 15 and 25° C. the reaction mixture is heated to a temperature being at least 45° C. Preferably, reaction mixture is heated to a temperature of at least 60° C., such as between 60° C. and 80° C. Typically, following heating of the reaction mixture, the reaction is continued for at least 4 hours, such as between 4 and 6 hours. Following this the reaction mixture is cooled to a temperature between 0° C. and 15° C., such as a temperature between 0° C. and 10° C., e.g about 5° C. Typically, in the reaction mixture the molar ratio between the compound of formula XVI and the reagent introducing a thiol or thiol linked to a protective group, such as TIPS-SH, Na₂S, or thiourea, is 1:2 to 2:1, preferably 2:3 to 3:2, e.g. about 1:1, and the organic solvent is in surplus.

The step b9 is a step for activating the anomeric position for nucleophilic substitution, to obtain the compound of formula XV wherein the anomeric position is alpha. The starting compound of formula XV

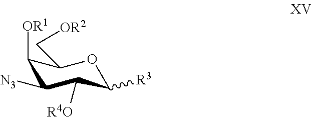

Wherein R1, R2, R3 and R4 are as defined above may be a mixture of the two anomers or may be the alpha or the beta anomer. Preferably, the compound of formula XV has R3 in the anomeric beta position. Typically, a halogen is introduced in the alpha anomeric configuration although or an ester or a thioester group may also be introduced. When a halogen is introduced to obtain the compound of formula XVI, the compound of formula XV is preferably halogenated using a halogenating agent such as BiBr₃, TMSBr, HBr and/or TiBr₄. The reaction takes place in a first suitable organic solvent with the halogenating agent in a second organic solvent. Typically, the first and second organic solvents are selected from polar aprotic solvents and may be the same or different. Preferred first and second organic solvents are selected from toluene, ethyl acetate, acetonitrile or DMF, typically, both are ethyl acetate or toluene. When brominating with hydrobromide the reaction solvent is usually acetic acid. Preferably the reaction takes place under an inert atmosphere, such as a nitrogen atmosphere. Preferably, the suitable temperature is between 15° C. and 70° C., such as from 20° C. to 65° C. In a further embodiment the suitable temperature is between 25° C. and 60° C., such as between 30° C. and 50° C. The reaction continues for at least 1 hour, such as for at least 4 hours, such as for at least 8 hours, such as for at least 16 hours such as between 1 and 20 hours. Hereafter the reaction mixture is cooled, such as to 20° C., or to 15° C. or lower. Preferably, the reaction mixture is cooled to a temperature between 0° C. and 15° C., such as about 5° C. in another embodiment the reaction mixture is cooled to a temperature between 50° C. and 20° C. Typically, in the reaction mixture the molar ratio between the compound of formula XV and the halogenating agent is 1:3 to 3:2, preferably 1:2 to 1:1, e.g. 3:4 and the organic solvent is in surplus.

In a second aspect the present invention relates to a compound of formula XVIII

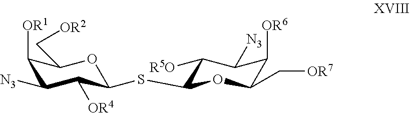

wherein R1, R2, R4, R5, R6 and R7 are as defined above. In one embodiment R1-R7 are all a protecting group. Typically, R1-R7 are the same protecting group, such as benzoyl, pivaloyl or acetyl. Preferably, R1-R7 are all acetyl groups.

In a third aspect the present invention relates to a compound of formula XIX

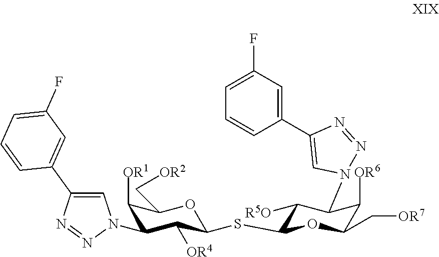

wherein R1, R2, R4, R5, R6 and R7 are as defined above. In one embodiment R1-R7 are all a protecting group. Typically, R1-R7 are the same protecting group, such as benzoyl, pivaloyl or acetyl. Preferably, R1-R7 are all acetyl groups.

In a fourth aspect the present invention relates to a compound of formula XVII

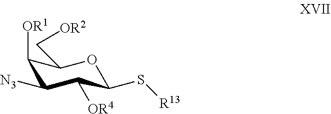

wherein R1, R2, and R4 are as defined above. Typically, R1, R2, R4 are the same protecting group, such as benzoyl, pivaloyl or acetyl. Preferably, R1, R2, R4 are all acetyl groups. R13 is preferably selected from the group consisting of hydrogen, a silyl group, e.g. triisopropylsilyl, a triflate group, acetyl, and C(═NH)—NH₂, typically C(═NH)—NH₂.

In a fifth aspect the present invention relates to a compound of formula XX

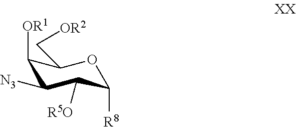

wherein R5, R6 and R7 are as defined above. Typically, R5, R6 and R7 are the same protecting group, such as benzoyl, pivaloyl or acetyl. Preferably, R5, R6 and R7 are all acetyl groups. Typically, R8 is a halogen or OR10 wherein R10 is selected from H, Z-C₁₋₆ alkyl, Z-C₂₋₆ alkenyl, Z—C₃₋₆branched alkyl, Z-C₃₋₆cyclo alkyl, Z-heteroaryl, and Z-aryl wherein Z is SO, SO₂, C═O or C═S. Preferably R8 is a halogen such as bromine.

In a sixth aspect the present invention relates to a compound of formula XVI

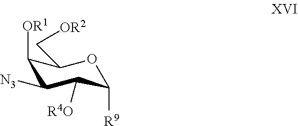

wherein R1, R2, and R4 are as defined above. Typically, R1, R2, R4 are the same protecting group, such as benzoyl, pivaloyl or acetyl. Preferably, R1, R2, R4 are all acetyl groups. Typically, R9 is a halogen or OR11 wherein R11 is selected from H, Z′-C₁₋₆ alkyl, Z′-C₂₋₆ alkenyl, Z′-C₃₋₆ branched alkyl, Z′-C₃₋₆ cyclo alkyl, Z′-heteroaryl and Z′-aryl wherein Z′ is SO, SO₂, C═O or C═S. Preferably R9 is a halogen such as bromine.

In a seventh aspect the present invention relates to a compound of formula XV

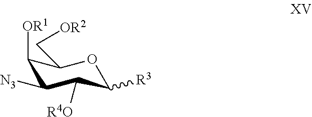

wherein the wavy line indicates that R3 is anomeric alpha, beta or a mixture of alpha and beta, preferably beta anomeric position, and wherein R1, R2, and R4 are as defined above. Typically, R1, R2, R4 are the same protecting group, such as benzoyl, pivaloyl or acetyl. Preferably, R1, R2, R4 are all acetyl groups. Typically, R3 is SR12 or OR12 wherein R12 is selected from H, Z″-C₁₋₆ alkyl, Z″-C₂₋₆ alkenyl, Z″-C₃₋₆ branched alkyl, Z″-C₋₆ cyclo alkyl Z″-heteroaryl and Z″-aryl wherein Z″ is SO, SO₂, C═O or C═S. Preferably, R3 is OR12, wherein R12 is as defined above, such as Z″-C₁₋₆ alkyl, Z″-C₋₆ branched alkyl, Z″-C₃₋₆ cyclo alkyl and Z″-phenyl wherein Z″ is C═O, e.g. acetyl. The compound of formula XV wherein R1, R2 and R4 are all acetyl and R3 is acetyl as a mixture of alpha and beta anomers have been disclosed in Lowary, T. L. and Hindsgaul, O. (1994) Recognition of synthetic O-methyl, epimeric, and amino analogues of the acceptor alpha-L-Fucp-(1,2-beta-D-Galp-OR by the blood group A and B gene-specified glycosyltransferases. Carbohydr. Res. 251: 33-67.

In a particular aspect the intermediate compound of formula XVII can be avoided during the process for preparing the compound of formula I when starting from the compound of formula XV or XVI.

Consequently, the present invention relates to a process for preparing 3,3′-Dideoxy-3,3′-bis-[4-(3-fluorophenyl)-1H-1,2,3-triazol-1-yl]-1,1′-sulfanediyl- di-β-D-galactopyranoside having formula (I)

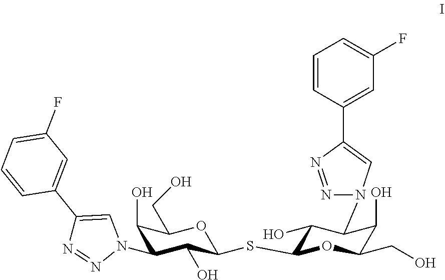

wherein the process comprises the consecutive steps of
(a) reacting a compound of formula XV

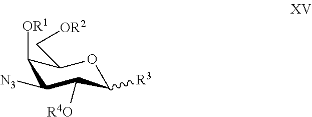

wherein the wavy line indicates that R3 is anomeric alpha, beta or a mixture of alpha and beta, wherein R1, R2, and R4 are independently selected from protecting groups or hydrogen, provided that at least one of R1, R2 and R4 is a protecting group, and R3 is a SR12 or OR12 wherein R12 is selected from H, Z″-C₁₋₆ alkyl, Z″-C₁₋₆ alkenyl, Z″-C₃₋₆branched alkyl, Z″-C₃₋₆ cyclo alkyl Z″-heteroaryl and Z″-aryl wherein Z″ is SO, SO₂, C═O or C═S, in a first suitable organic solvent with a reagent for activating the anomeric position for nucleophilic substitution in a second suitable organic solvent under suitable conditions to obtain the compound of formula XVI wherein R1, R2, and R4 are independently selected from protecting groups or hydrogen, provided that at least one of R1, R2, and R4 is a protecting group and R9 is a halogen,
b) reacting the compound of formula XVI

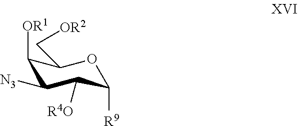

wherein R1, R2, and R4 are independently selected from protecting groups or hydrogen, provided that at least one of R1, R2, and R4 is a protecting group, and R9 is a halogen, with a sulphide reagent under suitable conditions to obtain the compound of formula XVIII wherein R1, R2, R4, R5, R6 and R7 are independently selected from protecting groups or hydrogen, provided that at least one of R1, R2, R4, R5, R6 and R7 is a protecting group,
c) reacting a compound of formula XVIII

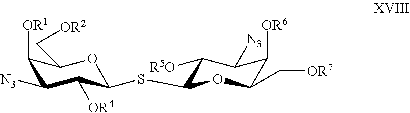

wherein R1, R2, R4, R5, R6 and R7 are independently selected from protecting groups or hydrogen, provided that at least one of R1, R2, R4, R5, R6 and R7 is a protecting group, with 3-fluorophenyl acetylene or a 3-fluorophenyl acetylene protected at the terminal position of the acetylene group, under suitable conditions to obtain a compound of formula XIX

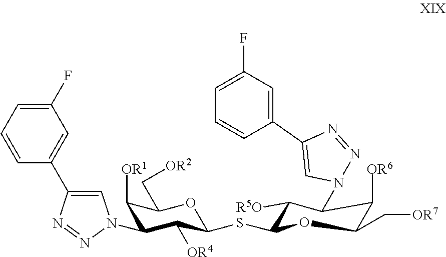

wherein R1, R2, R4, R5, R6 and R7 are as defined above, and d) removing the protecting groups of the compound of formula XIX to obtain the compound of formula I.

In an embodiment R1, R2, R4, R5, R6 and R7 are independently selected from ester protecting groups, such as acetyl, benzoyl and pivaloyl, typically all R1, R2, R4, R5, R6 and R7 are identical, such as acetyl.

In a further embodiment R3 in formula XV is in the beta anomeric position and is OR12 wherein R12 is CO-C₁₋₆ alkyl, preferably R12 is acetyl.

In a further embodiment R9 in formula XVI is a bromine or chlorine, such as bromine.

All the above described embodiments from step b9 are also considered individual embodiments for this step a).

In a still further embodiment the suitable conditions in step (a) are reacting a compound of formula XV wherein R1, R2, R3 and R4, are as defined above, optionally under inert atmosphere and at a suitable temperature with the halogenating agent in an organic solvent, and maintaining the reaction mixture at the suitable temperature and continue the reaction for at least 1 hour, such as 1-6 hours, until at least 98% v/v conversion of XV, followed by cooling to a temperature at least 15° C. lower than the suitable temperature, and optionally isolating and purifying to obtain the compound of formula XVI as an oil.

In a further embodiment the reaction takes place under inert atmosphere, such as a nitrogen atmosphere.

In a still further embodiment the organic solvent is selected from the group consisting of ethyl acetate, toluene, DMF and acetonitrile, such as toluene.

In a further embodiment the suitable temperature is between 20° C. and 65° C., such as 35-55° C.

In a still further embodiment the halogenating agent is selected from any one of BiBr₃, TMSBr, HBr and TiBr_{4,} such as TiBr₄.

In a further embodiment the reaction is continued for at least 2 hours, such as from 3-5 hours, at the suitable temperature.

In a still further embodiment the reaction mixture is cooled to a temperature between 10° C. and 30° C., such as between 15° C. and 25° C.

In a still further embodiment in step (b) reacting a solution of the compound of formula XVI with the sulphide reagent for sufficient time under adequate cooling below 15° C. until at least 98% v/v conversion of XVI.

In a further embodiment in step (b) the sulphide reagent is selected from the group consisting of a sulphide nucleophile and a sulphide nucleophile surrogate. Typically, the sulphide nucleophile is selected form one or more of sodium sulphide, sodium hydrosulphide, potassium sulphide, and lithium sulphide. Typically, the sulphide nucleophile surrogate is selected from one or more of thiourea, TIPS—SH, Me₃SiCH₂CH₂SH, thioacetamide, TMS-Si—S—H, TBDMSSi—SH and BnSH.

When the sulphide reagent is a sulphide nucleophile surrogate it may need a further reagent to convert it to the sulphide nucleophile and typically such additive is for instance TBAF for a silyl protecting group, or alternatively sodium meta bisulphite to convert the thiourea adduct to the sulphide.

In a still further embodiment in step (b) the reaction is continued for at least 20 hours, such as from 22-26 hours, at the cooling temperature. Preferably, the reaction is stirred vigorously for the at least 20 hours.

In a further embodiment in step (b) the cooling is to a temperature below 10° C., such as from 0° C. to 10° C., such as from 0° C. to 5° C.

In a still further embodiment in step (b) the compound of formula XVI is dissolved in an organic solvent. Typically, the solvent is selected from the group consisting of ethyl acetate, toluene, DMF and acetonitrile, such as acetonitrile.

In a further embodiment in step (b) the reaction mixture is degassed with vacuum nitrogen purge cycles prior to continuing reaction for at least 20 hours.

In a still further embodiment in step (c) reacting a solution of the compound of formula XVIII with 3-fluorophenyl acetylene or a 3-fluorophenyl acetylene protected at the terminal position of the acetylene group, a catalyst and a base under suitable conditions to obtain a compound of formula XIX.

In a further embodiment in step (c) the catalyst is a metal catalyst, such as a metal halide, e.g. Cu(I) or Cu(II), in particular Cu halide, such as Cu iodide.

In a still further embodiment in step (c) the base is an organic base, such as an aliphatic amine base, such as triethylamine and N,N-Diisopropylethylamine (DIPEA).

In a further embodiment in step (c) the organic solvent is selected from toluene or a polar aprotic solvent, such as acetonitrile or DMF, and mixtures thereof, preferably acetonitrile.

In a still further embodiment in step (c) reacting the solution of the compound of formula XVIII with 3-fluorophenyl acetylene or a 3-fluorophenyl acetylene protected at the terminal position of the acetylene group, and the catalyst at a temperature from 10° C. to 30° C., and then adding the base for a sufficient time and keeping the temperature lower than 60° C. until at least 98% v/v conversion of XVIII. Typically, the sufficient time is at least 2 hours, such as 3-5 hours.

In a further embodiment in step (d) the removing of protecting groups in step c) is done by mixing the compound of formula XIX with a base and reacting for at least 15 minutes at a suitable temperature, followed by neutralizing with a suitable acid in solution to obtain the compound of formula I. Further processes for removing the protecting groups are described in detail in the section Experimental 2, and further relevant embodiments are described above in relation to step b13, all of which are considered embodiments under this aspect of the present invention.

The compound of formula I may be obtained as a crystalline form after purification. Thus, in one embodiment the isolated crystalline compound of formula I is obtainable by dissolving the 3,3′-Dideoxy-3,3′-bis-[4-(3-fluorophenyl)-1H-1,2,3-triazol-1-yl]-1,1′-sulfanediyl-di-β-D-galactopyranoside in ethanol and purified water at 45±5 ° C. over 90 minutes; filtering the solution through a fine filter (0.45 μm) followed by a line rinse of ethanol; distilling the combined filtrates in vacuo at 35±5° C. and cooling the solution to 20±5° C.; adding ethanol and distilling the solution in vacuo at 35±5° C. and cooling the solution cooled to 20±5° C.; adding ethanol and distilling the solution in vacuo at 35±5° C. prior to confirming the water content of the slurry to be <7.6% w/w; hereafter heating the slurry to 70±5° C. for 1 hour then cooling to 20±5° C. over 1.5 hours and aging at this temperature for 18 hours; filtering the slurry at 20±5° C. and washing the filter cake with ethanol; drying the filter cake at 20±5° C. under a stream of air for 4 days to give 3,3′-Dideoxy-3,3′-bis-[4-(3-fluorophenyl)-1H-1,2,3-triazol-1-yl]-1,1′-sulfanediyl-di-β-D- galactopyranoside as a white to off white crystalline solid. More details regarding purification are explained in detail in the section Experimental 2.

The alpha and beta anomers of the compound of formula XV may be separated by various methods such as via crystallization. However, for the present process the starting point can be the mixture (the compound of formula XV) as well as one of the anomers.

The term “C₁₋₆ alkyl” as used herein means an alkyl group containing 1-6 carbon atoms, such as methyl, ethyl, propyl, butyl, pentyl or hexyl.

The term “C₂₋₆ alkenyl” as used herein means an alkenyl group containing 2-6 carbon atoms and at least one unsaturation, such as vinyl, allyl, butenyl, pentenyl, hexenyl.

The term “C₃₋₆ cycloalkyl” as used herein means a cyclic alkyl group containing 3-6 carbon atoms, such as cyclopropyl, cyclobutyl, cyclopentyl, cyclohexyl, cycloheptyl, and 1-methylcyclopropyl.

The term “aryl” as used herein means a phenyl or naphthyl.

The term “heteroaryl” as used herein means a mono or bicyclic aromatic ring system containing one or more heteroatoms, such as 1-10, e.g. 1-6, selected from 0, S, and N, including but not limited to oxazolyl, oxadiazolyl, thiophenyl, thiadiazolyl, thiazolyl, pyridyl, pyrimidinyl, pyridonyl, pyrimidonyl, quinolinyl, azaquionolyl, isoquinolinyl, azaisoquinolyl, quinazolinyl, azaquinazolinyl, bensozazoyl, azabensoxazoyl, bensothiazoyl, or azabensothiazoyl.

The term “a protective group” or “a protection group” as used herein means a group which is introduced into a molecule by chemical modification of a functional group to obtain chemo selectivity in a subsequent chemical reaction. In many preparations of the compounds as described herein, some specific parts of the molecules cannot survive the required reagents or chemical environments and consequently, these parts, or groups, must be protected. Examples of suitable protecting groups without limitation are ester protecting groups, such as acetyl, benzoyl and pivaloyl, silyl group, e.g. triisopropylsilyl, a triflate group, acetyl, or C(═NH)—NH₂.

The term “an organic solvent” as used herein means a carbon based solvent suitable for dissolving or partly dissolving a molecule thereby making it suitable for reaction with another molecule. Suitable organic solvents are without limitation selected from toluene, dichloro-methane, ethyl acetate, methanol, ethanol, acetonitrile, or DMF, and mixtures thereof. As used herein ‘first’, ‘second’, ‘third’ and ‘fourth’ organic solvent is independently selected from any organic solvent known to the skilled person, such as any one of the above.

The term “a base” as used herein means a chemical species that donates electrons, accepts protons, or releases hydroxide (OH—) ions in aqueous solution. Typical types of bases include Arrhenius base, Bronsted-Lowry base, and Lewis base. The “base” includes “an organic base” such as an aliphatic amine base, e.g. triethylamine or N,N-Diisopropylethylamine (DI-PEA), or stronger bases such as LDA, NaHMDS, pyridine, or DMAP.

The term “a reagent introducing a thiol or thiol linked to a protective group” as used herein means a reagent that contains a thiol group or a thio group, and which thiol or thio optionally is protected or masked, which reagent upon reaction with a decided position on a molecule introduces SH, or S-protection group, such as TIPS—SH, Na₂S, or thiourea.

The term “a reagent for activating the anomeric position for nucleophilic substitution” as used herein means a reagent that activates the anomeric position to enable nucleophilic substitution of a given position with a nucleophile such as a thiol. The product of such an activation could be a halide such as bromine or a sulfate ester such as a triflate. The activating agent as used herein means an agent for introducing a halogen, such as bromine.

The term “a sulphide reagent” as used herein means a sulphide nucleophile, such as but not limited to sodium sulphide, sodium hydrosulphide, potassium sulphide, lithium sulphide, or a sulphide nucleophile surrogate which is a masked sulphide nucleophile, such as but not limited to thiourea, TIPS-SH, Me₃SiCH₂CH₂SH, or thioacetamide, which can be converted to a sulphide nucleophile. The sulphide reagent is useful for nucleophilic displacement of the R⁹ halogen of compound of formula XVI leading to the subsequent transformation into compound of formula XVIII.

The term “treatment” and “treating” as used herein means the management and care of a patient for the purpose of combating a condition, such as a disease or a disorder. The term is intended to include the full spectrum of treatments for a given condition from which the patient is suffering, such as administration of the active compound to alleviate the symptoms or complications, to delay the progression of the disease, disorder or condition, to alleviate or relief the symptoms and complications, and/or to cure or eliminate the disease, disorder or condition as well as to prevent the condition, wherein prevention is to be understood as the management and care of a patient for the purpose of combating the disease, condition, or disorder and includes the administration of the active compounds to prevent the onset of the symptoms or complications. The treatment is performed in a chronic way. The patient to be treated is a human subject diagnosed with pulmonary fibrosis or other types of lung fibrosis.

The term “an amount effective to treat pulmonary fibrosis” of a compound of formula (I) of the present invention as used herein means an amount sufficient to cure, alleviate or partially arrest the clinical manifestations of pulmonary fibrosis and its complications. Effective amounts for each purpose will depend on the severity of the disease or injury as well as the weight and general state of the subject. It will be understood that determining an appropriate dosage may be achieved using routine experimentation, by constructing a matrix of values and testing different points in the matrix, which is all within the ordinary skills of a trained physician or veterinary.

As used herein “pharmaceutically acceptable additive” is intended without limitation to include carriers, excipients, diluents, adjuvant, colorings, aroma, preservatives etc. that the skilled person would consider using when formulating a compound of the present invention in order to make a pharmaceutical composition.

The adjuvants, diluents, excipients and/or carriers that may be used in the composition of the invention must be pharmaceutically acceptable in the sense of being compatible with the compound of formula (I) and the other ingredients of the pharmaceutical composition, and not deleterious to the recipient thereof. It is preferred that the compositions shall not contain any material that may cause an adverse reaction, such as an allergic reaction. The adjuvants, diluents, excipients and carriers that may be used in the pharmaceutical composition of the invention are well known to a person within the art.

As mentioned above, the compositions and particularly pharmaceutical compositions as herein disclosed may, in addition to the compounds herein disclosed, further comprise at least one pharmaceutically acceptable adjuvant, diluent, excipient and/or carrier. In one embodiment the pharmaceutical composition contains neat compound of formula I. In some embodiments, the pharmaceutical compositions comprise from 1 to 99 weight % of said at least one pharmaceutically acceptable adjuvant, diluent, excipient and/or carrier and from 1 to 99 weight % of a compound of formula I as herein disclosed. The combined amount of the active ingredient and of the pharmaceutically acceptable adjuvant, diluent, excipient and/or carrier may not constitute more than 100% by weight (100% w/w) of the composition, particularly the pharmaceutical composition.

In accordance with Controlled Pulmonary Drug Delivery, Smith and Hickey, Editors, Springer 2011 in particular chapters 13, 14 and 15 the skilled person will know how to formulate compounds, such as the compound of formula (I) for pulmonary drug delivery.

Dry powder inhalers (DPI), are well known for dispensing medicament to the lungs of a patient. Preferred DPIs for use in the present invention is a monodose dry powder inhaler from Plastiape (HQ, Osnago, Italy), in particular the RS01 Monodose Dry Powder Inhaler.

Further embodiments of the process are described in the experimental section herein, and each individual process as well as each starting material constitutes embodiments that may form part of embodiments.

The above embodiments should be seen as referring to any one of the aspects (such as ‘method for treatment’, ‘pharmaceutical composition’, ‘compound for use as a medicament’, or ‘compound for use in a method’) described herein as well as any one of the embodiments described herein unless it is specified that an embodiment relates to a certain aspect or aspects of the present invention.

All references, including publications, patent applications and patents, cited herein are hereby incorporated by reference to the same extent as if each reference was individually and specifically indicated to be incorporated by reference and was set forth in its entirety herein.

All headings and sub-headings are used herein for convenience only and should not be construed as limiting the invention in any way.

Any combination of the above-described elements in all possible variations thereof is encompassed by the invention unless otherwise indicated herein or otherwise clearly contradicted by context.

The terms “a” and “an” and “the” and similar referents as used in the context of describing the invention are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein. Unless otherwise stated, all exact values provided herein are representative of corresponding approximate values (e.g., all exact exemplary values provided with respect to a particular factor or measurement can be considered to also provide a corresponding approximate measurement, modified by “about,” where appropriate).

All methods described herein can be performed in any suitable order unless otherwise indicated herein or otherwise clearly contradicted by context.

The use of any and all examples, or exemplary language (e.g., “such as”) provided herein, is intended merely to better illuminate the invention and does not pose a limitation on the scope of the invention unless otherwise indicated. No language in the specification should be construed as indicating any element is essential to the practice of the invention unless as much is explicitly stated.

The citation and incorporation of patent documents herein is done for convenience only and does not reflect any view of the validity, patentability and/or enforceability of such patent documents.

The description herein of any aspect or embodiment of the invention using terms such as “comprising”, “having”, “including” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

This invention includes all modifications and equivalents of the subject matter recited in the aspects or claims presented herein to the maximum extent permitted by applicable law.

The present invention is further illustrated by the following examples that, however, are not to be construed as limiting the scope of protection. The features disclosed in the foregoing description and in the following examples may, both separately and in any combination thereof, be material for realizing the invention in diverse forms thereof.

### Experimental 1

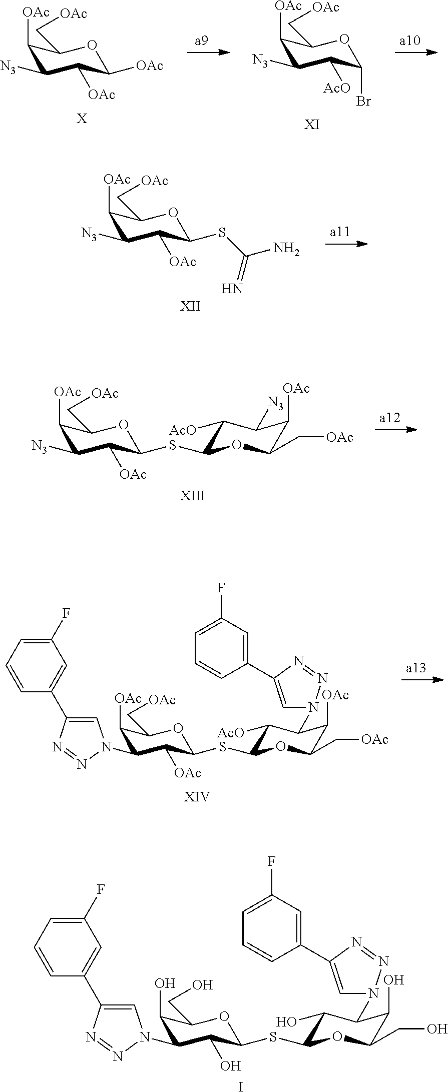

In above reaction scheme formula X is a known starting compound used for preparing formula (I). In the above scheme compound XII is a hydrobromide salt.

Here below is described a preferred embodiment using the thiourea route, and this is a preferred embodiment of the present invention:

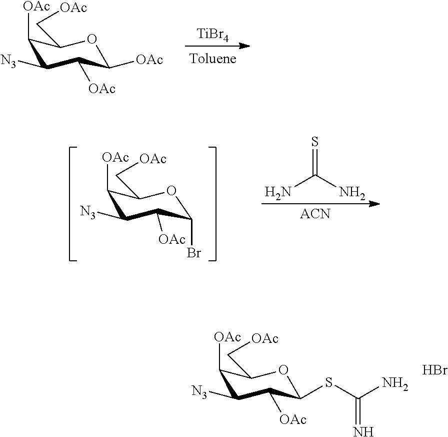

The compound of formula X, TiBr₄ (0.5 eq) and toluene are charged into a reaction. The stirring is started, and the mixture is heated to 45° C. The mixture is stirred at 45° C. for 4-5hrs and then cooled to 20° C. The mixture is filtered to remove solids and the filter cake is washed with toluene. The combined filtrates are washed with aqueous Ethylenediaminetetraacetic acid (EDTA) (38%) and aqueous sodium thiosulfate (10%). The organic phase is concentrated under vacuum to about 2 vol and then diluted with acetonitrile (ACN). Thiourea (1.1 eq) is added and the mixture is heated to 55° C. and stirred there until end of reaction (3-4 hrs). The resulting slurry is cooled to 10° C. over a period of at least 4 hrs. The product is isolated by filtration and the filter cake is washed with acetonitrile and dried under vacuum.

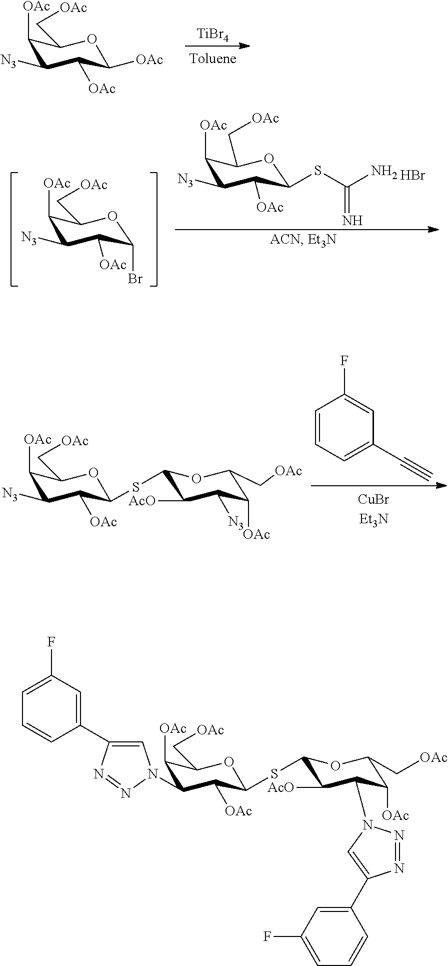

The compound of formula X, TiBr4 (0.5 eq) and toluene are charged into a reaction. The stirring is started, and the mixture is heated to 45° C. The mixture is stirred at 45° C. for 4-5hrs and then cooled to 20° C. The mixture is filtered to remove solids and the filter cake is washed with toluene. The combined filtrates are washed with aqueous EDTA (38%) and aqueous sodium thio-sulfate (10%). The organic phase is concentrated under vacuum to 2 vol and then diluted with acetonitrile. The compound of formula XII (0.96 eq) is added together with triethylamine (2.4-3.0 eq) and the mixture is stirred at 0-30° C. until end of reaction. The mixture is then filtered to remove NaBr and triethylamine (0.4 eq) and 3-fluorophenyl acetylene, are charged. The mixture is heated to 55° C. until end of reaction. Methanol is charged and the mixture stirred for one hour and then cooled to 20° C. The crude compound of formula XIV is isolated by filtration and the filter cake is washed with methanol. Here below is described a preferred embodiment using the Na₂S route, and this is another preferred embodiment of the present invention:

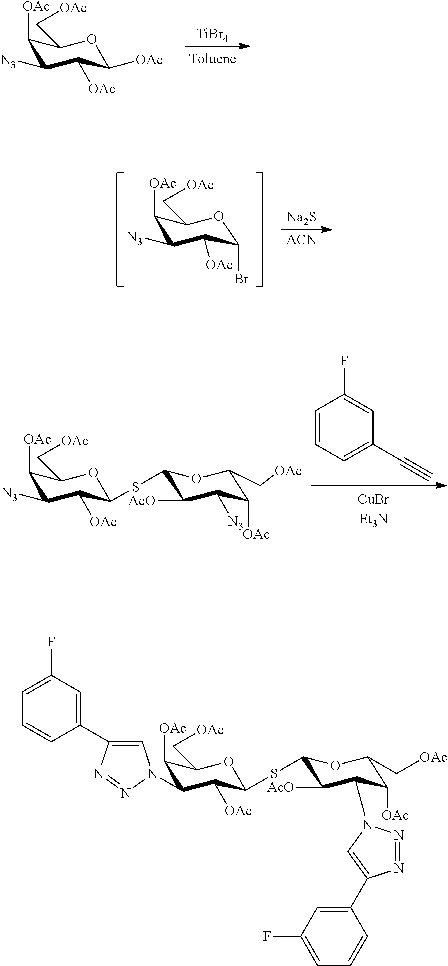

The compound of formula X, TiBr₄ (0.5 eq) and toluene are charged into a reactor. The stirring is started, and the mixture is heated to 45° C. The mixture is stirred at 45° C. for 4-5hrs and then cooled to 20° C. The mixture is filtered to remove solids and the filter cake is washed with toluene. The combined filtrates are washed with aqueous EDTA (38%) and aqueous sodium thiosulfate (10%). The organic phase is concentrated under vacuum to 2 vol and then diluted with acetonitrile. Na₂S (0.5 eq) is added and the mixture is stirred at −10-20° C. until end of reaction. The mixture is then filtered to remove NaBr then triethylamine (0.4eq) and 3-fluorophenyl acetylene (1.1eq) are charged. The mixture is heated to 55° C. until end of reaction. Methanol is charged and the mixture stirred for one hour and then cooled to 20° C. The crude compound of formula XIV is isolated by filtration and the filter cake is washed with methanol.

Different methods of deprotection are available, but one option is to recrystallize the compound of formula XIV using an acetone/methanol system here.

The current process to manufacture 3,3′-Dideoxy-3,3′-bis-[4-(3-fluorophenyl)-1H-1,2,3 -triazol-1-yl]-1,1′-sulfanediyl-di-β-D-galactopyranoside having formula I involves several process steps as described in detail hereunder.

### General Procedures

Nuclear Magnetic Resonance (NMR) spectra were recorded on a 400 MHz Bruker Avance AV400 spectrometer at 25° C. Chemical shifts are reported in ppm (δ) using the residual solvent as the internal standard. Peak multiplicities are expressed as follows: s, singlet; d, doublet; t, triplet; q, quartet; m, multiplet; br s, broad singlet.

The following abbreviations are used:
Ac: Acetyl
aq.: aqueous
DCM: Dichloromethane
DMF: N,N-Dimethylformamide
rt: room temperature
Sat.: Saturated
TBME: tert-Butylmethyl ether
TEMPO: (2,2,6,6-Tetramethylpiperidin-1-yl)oxyl
   2,4,6-Tri-O-acetyl-3-azido-3-deoxy-α-D-galactopyranosyl bromide, XI

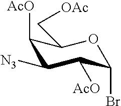

To a solution of TiBr₄ (1.28 kg, 3.48 mol) in ethyl acetate (2.24 kg) under argon was charged a solution of 1,2,4,6-Tetra-O-acetyl-3-azido-3-deoxy-β-D-galactopyranoside, X (1.00 kg, 2.68 mol) in ethyl acetate (1.80 kg) maintaining the temperature less than 25° C. using a jacketed vessel. A line rinse was conducted with ethyl acetate (0.45 kg). The reaction mixture was stirred at rt for 20 hours then cooled to 5° C. The mixture was washed with water (5 L) maintaining the temperature less than 25° C. using a jacketed vessel. The organic phase was washed sequentially with 5% aq. NaHCO₃ solution (2×4 L), 20% aq. Na₂S₂O₃ solution (4 L) and 10% aq. NaCl solution (4 L). The organic phase was dried over MgSO₄ (0.5 kg), filtered and concentrated in vacuo at 30° C. to yield 1.03 kg (98%) of 2,4,6-Tri-O-acetyl-3-azido-3-deoxy-α-D-galactopyranosyl bromide as an oil. ¹H NMR (400 MHz, CDCl₃) δ 6.70 (d, J=3.8 Hz, 1H), 5.49 (dd, J=3.3, 1.4 Hz, 1H), 4.94 (dd, J=10.6, 3.8 Hz, 1H), 4.41 (t, J=6.5 Hz, 1H), 4.18 (dd, J=11.5, 6.1 Hz, 1H), 4.15-4.00 (m, 2H), 2.17 (s, 3H), 2.15 (s, 3H), 2.06 (s. 3H).

2,4,6-Tri-O-acetyl-3-azido-1-carbamimidoylthio-3-deoxy-β-D-galactopyranoside hydrobromide, XII

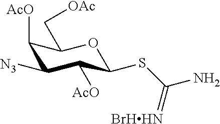

To a flask containing thiourea (316 g, 4.15 mol) and acetonitrile (4.95 kg) at rt under argon was charged a solution of 2,4,6-Tri-O-acetyl-3-azido-3-deoxy-α-D-galactopyranosyl bromide, XI (1.64 kg, 4.15 mol) in acetonitrile (2.4 kg). The reaction mixture was heated to 80° C. and stirred for 5 hours then cooled to 5° C. The mixture was stirred at 5° C.±5° C. for 5 hours then filtered, washing the precipitated material with pre-cooled (5° C.) acetonitrile (1.55 kg). The solid product was collected from the filter to give 1.18 kg (61%) of 2,4,6-Tri-O-acetyl-3-azido-1-carbamimidoylthio-3-deoxy-β-D-galactopyranoside hydrobromide as an off-white to grey solid. ¹H NMR (400 MHz, MeOD) δ 5.55 (dd, J=3.2, 0.7 Hz, 1H), 5.46 (d, J=10.1 Hz, 1H), 5.28 (t, J=10.1 Hz, 1H), 4.34 (ddd, J=7.0, 5.2, 0.9 Hz, 1H), 4.25-4.10 (m, 3H), 2.17 (s, 3H), 2.16 (s, 3H), 2.06 (s, 3H).

2,2′, 4,4′, 6,6′-Hexa-O-acetyl-3,3′-diazido-3,3′-dideoxy-1,1′-sulfanediyhdi-β-D-galactopyranoside, XIII

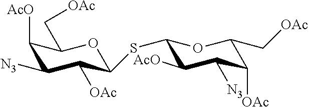

To a solution of 2,4,6-Tri-O-acetyl-3-azido-1-carbamimidoylthio-3-deoxy-β-D-galactopyranoside hydrobromide, XII (1.06 kg, 2.25 mol) in acetonitrile (2.49 kg) at 20° C. under argon was added a solution of 2,4,6-Tri-O-acetyl-3-azido-3-deoxy-α-D-galactopyranosyl bromide, XI (0.92 kg, 2.34 mol) in acetonitrile (2.49 kg). Additional acetonitrile (1.66 kg) and triethylamine (0.55 kg, 5.39 mol) were added and the reaction mixture was stirred for 18 hours at 20 ° C. The reaction mixture was concentrated in vacuo at 40° C. The residue was suspended in ethyl acetate (9.48 kg) at rt and 2M aq. HCl solution (6.36 L) was charged. The mixture was stirred vigorously at rt for 45 minutes to give complete dissolution. The phases were separated, and the organic phase was washed sequentially with 5% aq. NaHCO₃ solution (2×6.36 L) and saturated aq. NaCl solution (6.36 L) at rt. The organic phase was dried over MgSO₄ (0.5 kg), filtered and concentrated in vacuo at 40° C. to give 1.58 kg of crude material. The crude material was suspended in methanol (8.48 kg) and heated to 53 ° C. The mixture was stirred for 1 hour then cooled to 10° C. The product was isolated by filtration and washed with pre-cooled (5° C.-10° C.) methanol (2.26 kg). The filter cake was dried under vacuum to give 1.02 kg (77%) of 2,2′, 4,4′, 6,6′-Hexa-O-acetyl-3,3′-diazido-3,3′-dideoxy-1,1′-sulfanediyl-di-β-D-galactopyranoside as an off-white solid. ¹H NMR (400 MHz, CDCl₃) δ 5.47 (dd, J=3.3, 1.0 Hz, 2H), 5.17 (t, J=10.0 Hz, 2H), 4.79 (d, J=10.0 Hz, 2H), 4.15-4.08 (m, 4H), 3.84 (td, J=6.5, 1.1 Hz, 2H), 3.65 (dd, J=10.2, 3.6 Hz, 2H), 2.18 (s, 6H), 2.13 (s, 6H), 2.06 (s, 6H).

2,2′, 4,4′, 6,6′-Hexa-O-acetyl-3,3′-dideoxy-3,3′-bis-[4-(3-fluorophenyl)-1H-1,2,3-triazol-1-yl]-1,1′-sulfanediyl-di-β-D- galactopyranoside, XIV

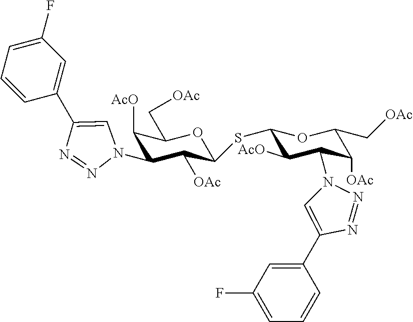

To a solution of 2,2′, 4,4′, 6,6′-Hexa-O-acetyl-3,3′-di-azido-3,3′-dideoxy-1,1′-sulfanediyl-di-β-D-galactopyranoside, XIII (0.39 kg, 0.59 mol) and CuI (22 g, 0.12 mol) in acetonitrile (6.13 kg) at rt under argon was added 3-fluorophenylacetylene (0.213 kg, 1.77 mol) and triethylamine (30 g, 0.30 mol). The reaction mixture was heated to 50° C. and stirred for 3 hours. The mixture was then concentrated in vacuo at 40° C. The residue was split into three portions and each portion was suspended in DCM (13.8 kg) and washed sequentially with 10% aq. NH₄OH solution (2×2.60 L), 20% aq. NaCl solution (2.60 L), 2 M aq. HCl (2×2.60 L) and 20% aq. NaCl solution (2.60 L) at rt. The solvent was removed from the combined organic phases by distillation at ambient temperature and the residue was then suspended in 1:1 methanol:DCM (6.83 L). The mixture was warmed to 40° C. and stirred for 20 minutes then cooled to 5° C. The mixture was filtered and washed with 1:1 methanol:DCM (0.78 L) at rt. The solid product was collected from the filter and dried under vacuum at 40° C. to give 0.48 kg (72%) of 2,2′, 4,4′, 6,6′-Hexa-O-acetyl-3,3′-dideoxy-3,3′-bis-[4-(3-fluorophenyl)-1H-1,2,3-triazol-1-yl]-1,1′-sulfanediyl-di-β-D- galactopyranoside as an off-white solid. ¹H NMR (400 MHz, DMSO-d₆) δ 8.78 (s, 2H), 7.80-7.64 (m, 4H), 7.56-7.46 (m, 2H), 7.25-7.13 (m, 2H), 5.74 (dd, J=10.8, 3.2 Hz, 2H), 5.64 (t, J=9.8 Hz, 2H), 5.49 (dd, J=3.3, 0.5, 2H), 5.27 (d, J=9.8 Hz, 2H), 4.39 (t, J=6.9 Hz, 2H), 4.21-4.02 (m, 4H), 2.09 (s, 6H), 2.07 (s, 6H), 1.88 (s, 6H). ¹⁹F NMR (376.5 MHz, DMSO-d₆) δ-112.63.

3,3′-Dideoxy-3,3′-bis-[4-(3-fluorophenyl)-1H-1,2,3-triazol-1-yl]-1,1′-sulfanediyl-di-β-D-galactopyranoside, I

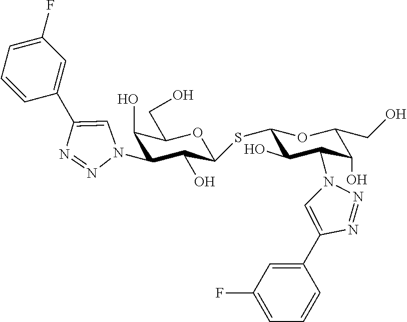

A suspension of 2,2′, 4,4′, 6,6′-Hexa-O-acetyl-3,3′-dideoxy-3,3′-bis-[4-(3-fluorophenyl)-1H-1,2,3-triazol-1-yl]-1,1′-sulfanediyl-di- β-D-galactopyranoside, XIV (0.46 kg, 0.52 mol) in acetonitrile (10.8 kg) was heated to 75° C. and stirred until dissolution was complete. The reaction mixture was then cooled to 45° C. and filtered. The filtrate was then stirred with 25 wt % sodium methoxide solution in methanol (0.435 kg) for 30 minutes at 20° C. The reaction mixture was then neutralised with 2 M aq. HCl solution (0.93 kg) and concentrated in vacuo at 50° C. The residue was suspended in methanol (0.525 kg), stirred and heated to 45° C. Water was added (7.28 kg) and the mixture was subsequently heated to 60° C. and stirred for 50 minutes. The suspension was cooled to 20° C., filtered and washed with water (1.67 kg). The solid was dried under vacuum at 60° C. then suspended in ethanol (1.31 kg). The mixture was heated to 70° C. and stirred for 30 minutes then cooled to 20° C. The solid was filtered, washed with ethanol (8×0.27 kg) then dried under vacuum at 70° C. to give 285 g (84%) of 3,3′-Dideoxy-3,3′-bis[4-(3-fluorophenyl)-1H-1,2,3-triazol-1-yl]-1,1′-sulfanediyl-di-β-D-galactopyranoside as a white to off-white solid. ¹H NMR (400 MHz, DMSO-d₆) δ 8.67 (s, 2H), 7.79-7.64 (m, 4H), 7.57-7.45 (m, 2H), 7.16 (tdd, J=8.7, 2.5, 0.7 Hz, 2H), 5.36 (br s, 4H), 4.95 (d, J=9.6, 2H), 4.87 (dd, J=10.5, 2.9 Hz, 2H), 4.70 (br s, 2H), 4.27 (t, J=10.5 Hz, 2H), 4.00 (bs, 2H), 3.75 (t, J=6.2 Hz, 2H), 3.66-3.47 (m, 4H). ¹⁹F NMR (376.5 MHz, DMSO-d₆) δ-112.76.

### Experimental 2

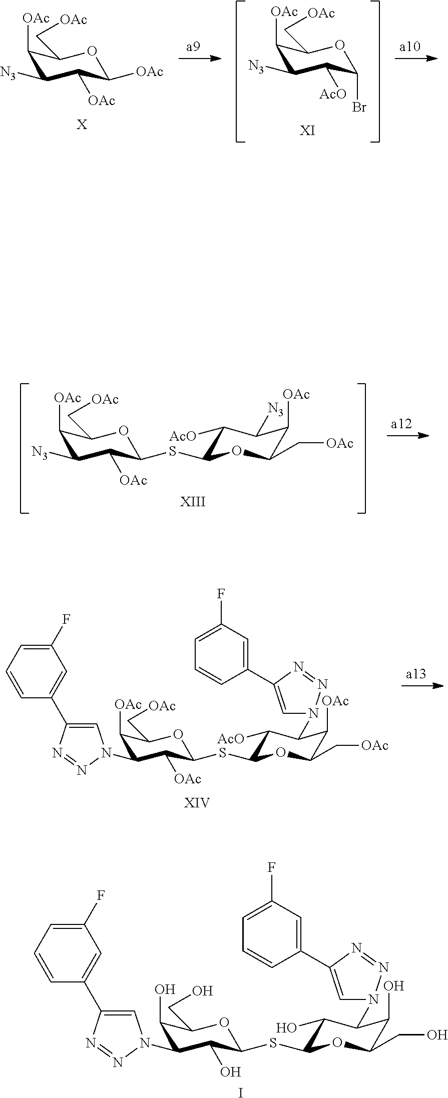

A further improved process to manufacture 3,3′-Dideoxy-3,3′-bis-[4-(3-fluorophenyl)-1H-1,2,3-triazol-1-yl]-1,1′-sulfanediyl-di-β-D-galactopyranoside having formula I involves several process steps as described in detail hereunder. The process has been modified to telescope several stages into one process thus converting the compound of formula (X) to the compound of formula (XIV) without isolating intermediates XI and XIII as shown in the reaction scheme above.

### General Procedures

Nuclear Magnetic Resonance (NMR) spectra were recorded on a 400 MHz Bruker Avance AV400 spectrometer at 25° C. Chemical shifts are reported in ppm (δ) using the residual solvent as the internal standard. Peak multiplicities are expressed as follows: s, singlet; d, doublet; t, triplet; q, quartet; m, multiplet; br s, broad singlet.

The following abbreviations are used:
Ac: Acetyl
aq.: aqueous
DCM: Dichloromethane
DMF: N,N-Dimethylformamide
rt: room temperature
Sat.: Saturated
TBME: tert-Butylmethyl ether

The following abbreviations are used:

2,2′, 4,4′, 6,6′-Hexa-O-acetyl-3,3′-dideoxy-3,3′-bis-[4-(3-fluorophenyl)-1H-1,2,3-triazol-1-yl]-1,1′-sulfanediyl-di-β-D- galactopyranoside, XIV

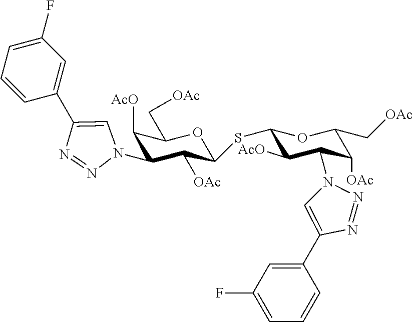

1,2,4,6- Tetra-O-acetyl-3-azido-3-deoxy-β-D-galactopyranoside, X 175 g, 469 mmol) and TiBr₄ (86 g, 234 mmol) were dissolved in toluene (1058 g), under nitrogen, and the mixture heated to 45±5° C. for 4 hours until <2% area X remained by HPLC analysis. The reaction mixture was cooled to 20±5° C., filtered and the filter cake washed with toluene (2×300 g). The combined filtrates were washed with 19% w/w aq. Na4EDTA solution (459 g, 234 mmol), then 15.7% w/w aq. Na₂S₂O₃ x5H₂O solution (370 g, 234 mmol) at 20±5° C. The phases were separated, and the organic phase distilled in vacuo to ca. 180 g at <50° C. prior to addition of acetonitrile (2059 g). The acetonitrile solution of 2,4,6-Tri-O-acetyl-3-azido-3-deoxy-α-D-galactopyranosyl bromide, XI was cooled to 0±5° C. and anhydrous Na₂S (31.1 g, 398 mmol) added. The mixture was degassed with vacuum nitrogen purge cycles, prior to stirring vigorously for 24 hours at 0±5° C. until <2% area XI remained by HPLC analysis. The mixture was filtered, the filter cake washed with acetonitrile (2×192 g) and the combined filtrates were distilled in vacuo to ca. 880 mL at <20° C. to yield an acetonitrile solution of 2,2′, 4,4′, 6,6′-Hexa-O-acetyl-3,3′-di-azido-3,3′-dideoxy-1,1′-sulfanediyl-di-β-D-galactopyranoside, XIII. 3-Fluorophenylacetylene (56.3 g, 469 mmol) was added followed by Cu(I)Br (5.04 g, 35.2 mmol) at 20±5° C. Triethylamine (23.7 g, 234 mmol) was added over >30 minutes at <55° C., the reaction then heated to 57±5° C. and held for 3 hours until <2% area of the monotriazole and <2% area of XIII were observed by HPLC analysis. 36% w/w HCl (24.2 g, 239 mmol) was added to adjust the pH to 4-5 then methanol (1750 g) over 2 hours at 55±5° C. The resulting slurry was cooled to 5±5° C. at a constant rate over 4 hours and held at 5±5° C. for 10 hours prior to filtration. The filter cake was washed with methanol (2×415 g) then dried at up to 40 ° C. to give 145 g, 161 mmol (yield: 68%) of 2,2′,4,4′,6,6′-Hexa-O-acetyl-3,3′-dideoxy-3,3′-bis-[4-(3-fluorophenyl)-1H-1,2,3-triazol-1-yl]-1,1′-sulfanediyl-di-β-D-galactopyranoside XIV as a grey to beige solid. ¹H NMR (400 MHz, DMSO-d₆) δ 8.78 (s, 2H), 7.80-7.64 (m, 4H), 7.56-7.46 (m, 2H), 7.25-7.13 (m, 2H), 5.74 (dd, J=10.8, 3.2 Hz, 2H), 5.64 (t, J=9.8 Hz, 2H), 5.49 (dd, J=3.3, 0.5, 2H), 5.27 (d, J=9.8 Hz, 2H), 4.39 (t, J=6.9 Hz, 2H), 4.21-4.02 (m, 4H), 2.09 (s, 6H), 2.07 (s, 6H), 1.88 (s, 6H). ¹⁹F NMR (376.5 MHz, DMSO-d₆) δ-112.63.

3,3′-Dideoxy-3,3′bis-[4-(3-fluorophenyl)-1H-1,2,3-triazol-1-yl]-1,1′-sulfanediyhdi-β-D-galactopyranoside, I [TD139-crude]

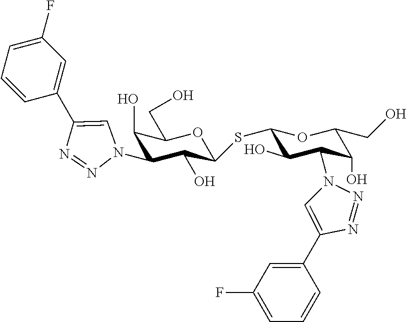

A suspension of 2,2′, 4,4′, 6,6′-Hexa-O-acetyl-3,3′-dideoxy-3,3′-bis-[4-(3-fluorophenyl)-1H-1,2,3-triazol-1-yl]-1,1′-sulfanediyl-di- β-D-galactopyranoside, XIV (130 g, 144 mmol) in methanol (1033 g, 11 vol) was degassed with vacuum nitrogen purge cycles then heated to 33±5° C. and stirred for 30 minutes. 30 wt % Sodium methoxide solution in methanol (156 g, 866 mmol) was degassed with vacuum nitrogen purge cycles then charged to the reaction over 15 to 45 minutes maintaining a reaction temperature of 33±5° C., followed by a degassed methanol line rinse (102 g, 1 vol). The reaction mixture was stirred at 35±5° C. for 3 hours and checked by HPLC analysis for reaction completion (limits <0.1 DEX283, <0.1 RRT 1.39). Acetic acid (53 g, 888 mmol) was charged to adjust the pH to 6 to 7 at 20±5° C. then activated charcoal (13 g) added and the mixture stirred for 1 hour at 35±5° C. The reaction was filtered and the filter washed with methanol (206g, 2 vol) at 35 ±5 ° C. The combined filtrates were treated with SiliaMetS® Thiourea (13 g) for 20 hours at 35±5° C. then filtered and the filter cake washed with methanol (103 g, 1 vol). The combined filtrates were distilled in vacuo to 835 mL, 6.5 vol, with a maximum jacket temperature of <49° C. then heated to 57±5° C. Degassed purified water (650 g, 5 vol) was added maintaining the temperature at 57±5° C. over ca. 0.5 hours. Additional 65 g water was charged over 5 minutes, which started the crystallization. The mixture was stirred at 57±5° C. until crystallization had become established (ca. 10 minutes). Further degassed purified water (1365g, 10.5 vol) was charged over ca. 2 hours at 57±5° C. then the slurry was cooled to 20±5° C. over 1.5 hours and held at this temperature for a further 15 hours. The solid was filtered, washed with purified water (520 g, 4 vol) then dried under vacuum at 40° C. to give 90,6 g, 140 mmol (92% assay corrected yield) of crude 3,3′-Dideoxy-3,3′-bis-[4-(3-fluorophenyl)-1H-1,2,3-triazol-1-yl]-1,1′-sulfanediyl-di-β-D-galactopyranoside as an off-white to light beige solid. ¹H NMR (400 MHz, DMSO-d₆) δ 8.67 (s, 2H), 7.79-7.64 (m, 4H), 7.57-7.45 (m, 2H), 7.16 (tdd, J=8.7, 2.5, 0.7 Hz, 2H), 5.36 (br s, 4H), 4.95 (d, J=9.6, 2H), 4.87 (dd, J=10.5, 2.9 Hz, 2H), 4.70 (br s, 2H), 4.27 (t, J=10.5 Hz, 2H), 4.00 (bs, 2H), 3.75 (t, J=6.2 Hz, 2H), 3.66-3.47 (m, 4H). ¹⁹F NMR (376.5 MHz, DMSO-d₆) δ 112.76.

3,3′-Dideoxy-3,3′-bis[4-(3-fluorophenyl)-1H-1,2,3-triazol-1-yl]-1,1′-sulfanediyl-di-β-D-galactopyranoside, I [TD139]

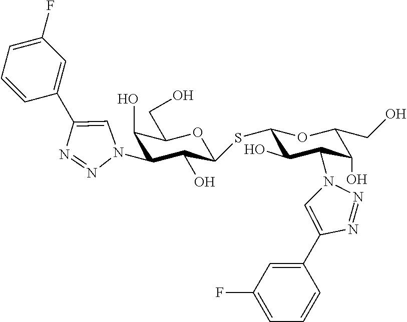

Crude 3,3′-Dideoxy-3,3′-bis-[4-(3-fluorophenyl)-1H-1,2,3-triazol-1-yl]-1,1′-sulfanediyl-di-β-D-galactopyranoside (89.4g, 138 mmol) was dissolved in ethanol (438 mL) and purified water (189 mL) at 45±5 ° C. over 90 minutes. The solution was filtered through a fine filter (0.45 μm) followed by a line rinse of ethanol (179 mL). The combined filtrates were distilled in vacuo. to 450 mL at 35±5° C. and the solution cooled to 20±5 ° C. Ethanol (893 mL) was added and the solution distilled in vacuo.to 450 mL at 35±5° C. and the solution cooled to 20±5° C.

Further ethanol (893 mL) was added and the solution distilled in vacuo.to 450 mL at 35±5° C. prior to confirming the water content of the slurry to be <7.6% w/w. The slurry was heated to 70±5 ° C. for 1 hour then cooled to 20±5 ° C. over 1.5 hours and aged at this temperature for 18 hours. The slurry was filtered at 20±5 ° C. and the filter cake washed with ethanol (541 mL). The filter cake was dried at 20±5 ° C. under a stream of air for 4 days to give 79.0 g, 117 mmol, 85% assay corrected yield of 3,3′-Dideoxy-3,3′-bis-[4-(3-fluorophenyl)-1H-1,2,3-triazol-1-yl]-1,1′-sulfanediyl-di-β-D-galactopyranoside as a white to off white crystalline solid.

1-68. (canceled)

69. A process suitable for large scale synthesis for preparing 3,3′-Dideoxy-3,3′-bis-[4-(3-fluorophenyl)-1H-1,2,3-triazol-1-yl]-1,1′-sulfanediyl-di-(3-D-galactopyranoside having formula (I) 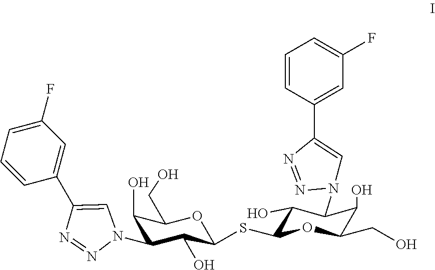
wherein the process comprises the consecutive steps of
a) reacting a compound of formula XVIII 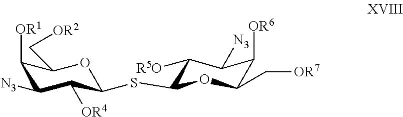
wherein R1, R2, R4, R5, R6 and R7 are independently selected from protecting groups or hydrogen, provided that at least one of R1, R2, R4, R5, R6 and R7 is a protecting group, with 3-fluorophenyl acetylene or a 3-fluorophenyl acetylene protected at the terminal position of the acetylene group, under suitable conditions to obtain a compound of formula XIX 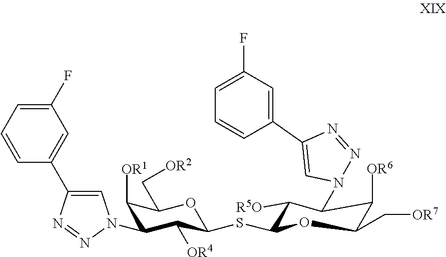
wherein R1, R2, R4, R5, R6 and R7 are as defined above, and
b) removing the protecting groups of the compound of formula XIX to obtain the compound of formula I.

70. The process of claim 69 wherein the compound of formula I is obtained as a crystalline or amorphous product.

71. The process of claim 69 wherein the suitable conditions in step a) are reacting a compound of formula XVIII wherein R1, R2, R4, R5, R6 and R7 are independently selected from protecting groups or hydrogen, provided that at least one of R1, R2, R4, R5, R6 and R7 is a protecting group, and a catalyst in an organic solvent optionally under inert atmosphere and at a suitable temperature with 3-fluorophenyl acetylene or a 3-fluorophenyl acetylene protected at the terminal position of the acetylene group, and optionally a base in the organic solvent to create a reaction mixture and optionally heating the reaction mixture to raise the temperature at least 15° C. above the suitable temperature and continue the reaction for at least 1 hour to obtain a compound of formula XIX wherein R1, R2, R4, R5, R6 and R7 are as defined above.

72. The process of claim 69 wherein R1, R2, R4, R5, R6 and R7 are independently selected form ester protecting groups, such as acetyl, benzoyl and pivaloyl, typically all R1, R2, R4, R5, R6 and R7 are identical, such as acetyl.

73. The process of claim 71 further comprising purifying and isolating the compound of formula XIX as a solid.

74. The process of claim 71 wherein the reaction takes place under inert atmosphere.

75. The process of claim 71 wherein the organic solvent is selected from toluene or a polar aprotic solvent, and mixtures thereof.

76. The process of claim 71 wherein the suitable temperature is between 15 and 25° C.

77. The process of claim 71 wherein the temperature is raised in the reaction mixture heating the mixture to 45° C. to 60° C.

78. The process of claim 71 wherein the reaction is continued for at least 2 hours.

79. The process of claim 71 wherein the catalyst is a metal catalyst.

80. The process of claim 71 wherein the base is an organic base.

81. The process of claim 69 wherein the removing of protecting groups in step b) is done by mixing the compound of formula XIX with a base and reacting for at least 15 minutes at a suitable temperature, followed by neutralizing with a suitable acid in solution to obtain the compound of formula I.

82. The process of claim 81 wherein the heating of the suspension is to at least 60° C.

83. The process of claim 81 wherein the base is in dissolved in an organic solvent.

84. The process of claim 81 wherein the base is selected from a base, in a concentration sufficient to provide deprotection.

85. The process of claim 81 wherein the base is sodium methoxide in methanol.

86. The process of claim 81 wherein the reaction with a base is for 20-60 minutes.

87. The process of claim 81 wherein the suitable temperature is 15-25° C.

88. The process of claim 81 wherein the neutralizing acid in solution is aqueous HCl.

89. The process of claim 71 wherein the molar ratio between the compound of formula XVIII and 3-fluorophenyl acetylene or a 3-fluorophenyl acetylene protected at the terminal position of the acetylene group is 1:5 to 1:1, and the organic solvent is in surplus.

90. The process of claim 89 wherein the molar ratio between the compound of formula XVIII and the catalyst is 3:1 to 7:1, and the organic solvent is in surplus.

91. The process of claim 89 wherein the molar ratio between the compound of formula XVIII and the base is 1:2 to 4:1, and the organic solvent is in surplus.

92. The process of claim 69 comprising a step directly preceding step a)
(i) reacting a compound of formula XVII 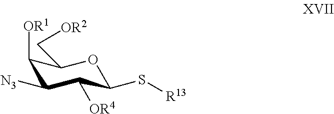
wherein R1, R2, and R4 are independently selected from protecting groups or hydrogen, provided that at least one of R1, R2, and R4 is a protecting group, and R13 is H or a protecting group, with a compound of formula XX 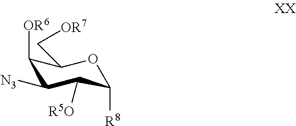
wherein R5, R6 and R7 are independently selected from protecting groups or hydrogen, provided that at least one of R5, R6 and R7 is a protecting group, and R8 is a halogen, SR10 or OR10 wherein R10 is selected from H, Z-C₁₋₆ alkyl, Z-C₁₋₆alkenyl, Z-C₃₋₆branched alkyl, Z-C₃₋₆cyclo alkyl, Z-heteroaryl, and Z-aryl wherein Z is SO, SO₂, C═O or C═S, under suitable conditions to obtain the compound of formula XVIII 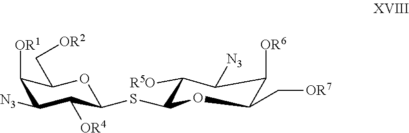
wherein R1, R2, R4, R5, R6 and R7 are independently selected from protecting groups or hydrogen, provided that at least one of R1, R2, R4, R5, R6 and R7 is a protecting group.
